SEQUENCE LISTING (19 sequences)

SEQ ID NO: 1 - protein, 279 aa, Triticum aestivum
MMHRADGLDLGLGLGLGLASQGSITSSTTTAASSSPASASHSQHWTAALSSVIGLRKEEPGVQASTSPESGGTKRGLERTGSGLAAGSDEDDDGGDGTGGRKKLRLSKDQAAVLEECFKTHSTLNPKQKTALANRLGLRPRQVEVWFQNRRARTKLKQTEVDCEYMKRCCEQLAEQNRRLEKEVAELRALKAAPPAHSAAAAAGPLTTLTMCLSCKRVASTSSASACDVPNFSTNAGMGMPMPSPVALPDHRQFFCGYRDTGATYGGFSGLAKVVKPAR

SEQ ID NO: 2 - DNA, 1222 bp, Triticum aestivum
gctcagaccagatcattacatcaccaccaccgagcactgccaccgtacgaaccatcgtccatcctcccagtccggggggcgcactctcagatcggatcgggtacgccggagacaagagacgatgatgcatcgcgccgacggcctcgacctgggcctcgggctggggctcggcctcgcttcccagggcagcatcacgtcctccaccaccaccgccgcatcctcctccccggcctccgcctcccactcgcagcactggaccgccgcgctcagctccgtcatcggcctgcggaaggaggaaccgggggtgcaggcttccacgtcgcccgagagcggcggcacaaagaggggcctggaacggaccggctccggcctggccgcgggcagcgacgaggacgacgacggcggtgatggcaccggcgggcgcaagaagctcaggctctccaaggaccaggccgccgtcctcgaggaatgcttcaagacgcacagcacgcttaaccccaagcagaagacggcgctggcgaaccggctgggcctccggccgcggcaggtggaggtgtggttccagaaccgccgggcgcgcaccaagctcaagcagacggaggtggactgcgagtacatgaagcgctgctgcgagcagctcgccgagcagaaccgccgcctcgagaaggaggtagccgagctccgcgcgctcaaggccgcgccgccggcgcacagcgcggcagcagcggcggggccactgaccaccctcaccatgtgcctctcctgcaagcgcgtcgcgtccacgtcttctgcctcggcctgcgacgtgcccaacttttccactaatgccggcatgggcatgcccatgccatccccggtggcactgccagaccaccggcagtttttttgcgggtacagagacaccggagcaacgtacggcggcttttcgggcctcgccaaggtggtcaagccggccagatagaacgagctatttctgtgacgccgccatgatcagcatgccaagcaagctataacaaaaatggcggcattgtcacaactcacaaacttaagctggtttagcagaaagtttagtccttcgttttctcagtctcagggggaaatgtaagttaattaattctagcaatttagggccgaattatccatcgaaatgagttcccggcggggatggatgtatgaatgtgaaaattaatgaaatgacatgagaaataattccccactcaaa

SEQ ID NO: 3 - DNA, 21 bp, Artificial
  oligonucleotide primer
gtacagagacaccggagcaac

SEQ ID NO: 4 - DNA, 21 bp, artificial
  oligonucleotide primer
ttgccaaatgtttgaacgatc

SEQ ID NO: 5 - DNA, 21 bp, artificial
  oligonucleotide primer
tcattgccagagtgttgtcag

SEQ ID NO: 6 - DNA, 21 bp, artificial
  oligonucleotide primer
ctaggctttatttagcgatac

SEQ ID NO: 7 - DNA, 21 bp, artificial
  oligonucleotide primer
ttcctccccctcccgaagatc

SEQ ID NO: 8 - DNA, 21 bp, artificial
  oligonucleotide primer
aagaacgtatttccgctattc

SEQ ID NO: 9 - DNA, 20 bp, artificial
  oligonucleotide primer
gcccaagggacccattctta

SEQ ID NO: 10 - DNA, 20 bp, artificial
  oligonucleotide primer
ttagaacttggaacccccca

SEQ ID NO: 11 - DNA, 21 bp, artificial
  oligonucleotide primer
tgagcagctgccgttgcttgg

SEQ ID NO: 12 - DNA, 23 bp, artificial
  oligonucleotide primer
aatagtagcctacatcacctctg

SEQ ID NO: 13 - DNA, 25 bp, artificial
  oligonucleotide primer
acagtttggacgcaagttttgtatt

SEQ ID NO: 14 - DNA, 25 bp, artificial
  oligonucleotide primer
cggtcctctgttcaattcttgttta

SEQ ID NO: 15 - DNA, 22 bp, artificial
  oligonucleotide primer
tgtggcatcaactgctaggaaa

SEQ ID NO: 16 - DNA, 23 bp, artificial
  oligonucleotide primer
cgtacaaagtgcctcataggaaa

SEQ ID NO: 17 - DNA, 20 bp, artificial
  oligonucleotide primer
acacgagtcactgggccaga

SEQ ID NO: 18 - DNA, 23 bp, artificial
  oligonucleotide primer
ctggtaaactagtcacccgctga

SEQ ID NO: 19 - DNA, 2814 bp, Hordeum vulgare
gaagagttgaatgatgaatccgggaagcccatctggaagaacagggttgacagttggagagagaagaagaacgagaagaaagcctcagcgaaaaaggctgcggctaaagcacaggttccgcctgcggaagaacagatcatggaagaaaaggagttgacagatgcgtatgagccactttcccggatcatccctatatcgaagacaagctcacacccttacagggcagtcatcattatgcggctagtagttctcgggcttttcttccactaccgtatcaccaaccctgtcgacagcgcctttggtctctggctgacttcagtcatatgtgagatctggttcggtttctcctggatcttggatcaacttcccaagtggtgtcctgtcaaccgagagacttatgttgataggctgattgcacgatatggagacggagaagattctgggttagcacctgtggatttctttgtcagtacagtggatccattgaaagagcctccgctaatcactgccaacactgtgctgtccattcttgcggtggactatcctgttgagaaaatttcgtgctacgtctctgacgatggatctgctatgctcacgtttgaatcacttgctgaaactgcggaatttgcaaggagatgggttccgttttgcaagaagttctccattgagccacgaacccctgagttctacttctcacagaagattgactacttgaaggataaaatacacccgtctttcgtcaaggagcgtagggctatgaagagagattatgaagagttcaaggtgaggataaatgctttggttgccaaggctcagaagacacctgaggaaggctgggtcatgcaggatgggacaccatggcctgggaacaactcccgtgaccaccccggcatgattcaggttttccttggtgagaccggtgctcgcgactacgatggaaatgagcttcctcggctagtatatgtttcaagagagaaaagaccagggtatcaacaccacaagaaggcaggggctatgaacgctctggtccgagtgtctgctgttctgacaaatgctccctacattcttaaccttgactgtgatcactatgttaacaacagcaaagctgtgcgtgaagcaatgtgcttcatgatggacccttctgttggtagagatgtctgctatgtccagttcccacagagattcgatgggattgatcgcagtgatcgttatgccaataggaacgtcgtgttctttgatcttatcgataccgtcaacatgaaagggcttgatggcatccaaggaccggtttatgtgggaactggttgttgtttctataggcaagcactctatggttatgggccaccatctctgcctgcccttccaaaatcttcggcttgttcgttctgctgctgctgtcctaagaacaaggttgaaaaaactgagaaagaaatgcacagagactccagacgagaagaccttgagtctgccattttcaatctacgggaaatcgacaactacgacgagtatgagcggtccatgcttatctcccagatgagcttcgagaagtcatttggacagtcttcggtattcattgaatcaacccttatggagaatggaggtgttcctgagtctgcagacccttctacactgattaaagaagccattcatgtcattagttgtgggtatgaagagaaaactgaatggggaaaagagcttggttggatctatggttcagttacagaggatattctgactgggtttaagatgcattgccgtgggtggcgatccatctactgcatgccgataagacccgcattcaagggatcagccccaatcaacctttcagatcgtttgcaccaggttcttcggtgggctctcggttctgtcgagatcttcttcagccggcattgcccgctgtggtatggctacggcggtggacgtctgagatggctccagaggctgtcctacatcaacaccatcgtctaccccttcacctctgttcctctcgtcgcatactgctgcctacctgccatttgcttgctcaccggcaaattcatcattcccatcctctccaatgccgcgacgatatggtttcttggcctcttcacctccatcatcctgacgagcgtcctggagctgcggtggagcggcattggcatcgaggactggtggcgcaacgagcagttctgggtcatcggcggcgtatctgctcatctctttgccgtgttccagggtatcctcaagatggtgatcgggctggacaccaacttcacggtcacgagcaaggccgcagaggacggcgatttcgctgagctgtatgtgttcaagtggacgacggtgctgatcccgcctacgacaatccttggtgctcaacctcgtttggcgtggtggccgggttcttcggacgcgctcaacagcggctacgagtcatgggggccgctcttcggcaaggtgttcttctccatgtgggtgattatgcacctgtacccgttcctcaagggtctcatgggccggcagaaccggactcccaccatcgtcatactctggtccgtcctgctggcctcggtcttctccctcctctgggtcaagatcgacccctttatcagcggcgccgagaccgtcgccaccggagcctgcagcagcatcgactgctgagcagctgtcattgcttggagttggaggtccaggcaattgtttgctggttagcggatgtgtgtatttgatcaatgtaaagaccgttggtatcctctcaggcctttgtccaccatttcggaggtgatgtaggctactattttgggtcgattatttcattgctgttgactctgttaaaaa

(19) United States
(12) Patent Application Publication
(10) Pub. No.: US 2010/0242140 A1
(43) Pub. Date: Sep. 23, 2010

(54) Modulation Of Plant Cell Wall Deposition

(76) Inventors: Sergiy Lopato, Morphett Vale (AU); Nataliya Kovalchuk, Brompton (AU)

Correspondence Address: BROWN & MICHAELS, PC;400 M & T BANK BUILDING, 118 NORTH TIOGA ST, ITHACA, NY, 14850, US

(21) Appl. No.: 12/738,650

(22) PCT Filed: Oct. 17, 2008
(86) PCT No.: PCT/AU08/01539

§ 371 Date: Jun. 3, 2010

(30) Foreign Application Priority Data
Oct. 19, 2007 (AU) 2007905748

(51) Int. Cl. A01H 5/00; C12N 5/04; C12N 5/10; C12Q 1/02; A61K 49/00; G01N 33/53

(52) U.S. Cl. 800/295; 435/410; 435/419; 435/29; 424/9.1; 435/7.2

(57) ABSTRACT

The present invention is predicated, in part, on the functional characterisation of a homeodomain/leucine zipper (HD-Zip) polypeptide which modulates various aspects of cell wall deposition in plant cells, including secondary cell wall deposition. The present invention provides, among other things, methods for modulating cell wall deposition in plant cells; plant cells and plants having modulated cell wall deposition; and methods for determining and/or predicting the rate and/or extent of cell wall deposition in plant cells and plants.

### PRIORITY CLAIM

The present application claims priority to Australian provisional patent application 2007905748 filed 19 Oct. 2007, the contents of which is hereby incorporated by reference.

### TECHNICAL FIELD

The present invention is predicated, in part, on the functional characterisation of a homeodomain/leucine zipper (HD-Zip) polypeptide which modulates various aspects of cell wall deposition in plant cells, including secondary cell wall deposition. The present invention provides, among other things, methods for modulating cell wall deposition in plant cells; plant cells and plants having modulated cell wall deposition; and methods for determining and/or predicting the rate and/or extent of cell wall deposition in plant cells and plants.

### BACKGROUND

The composition and structure of plant cell walls, including the extent of secondary cell wall deposition, has a dramatic impact on the value and utility of plant-derived raw materials such as wood, fibres, forage stock or other plant-biomass containing products.

The composition and structure of the plant cell wall can also have a significant impact on the agronomic traits and/or agronomic performance of a cultivated plant such as a cereal crop plant. For example, the extent of secondary cell wall deposition and/or cell wall strength in a plant can have a significant impact on agronomic traits such as stem strength and susceptibility to lodging.

In addition, a significant proportion of terrestrial plant biomass comprises lignified cell walls (including secondary cell walls). Thus, it would also be desirable if this large reservoir of carbon could be readily exploited for the production of chemicals and energy. For example, it would be desirable if the composition of plant cell walls, including the extent of secondary cell wall deposition, could be modulated such that the resultant plant biomass was particularly suitable for downstream conversion processes, such as the production of bioethanol; suitable for the exploitation of microorganisms and/or microbial enzymes for biomass pretreatments; or for the production of novel chemicals.

In light of the above, it would be desirable to provide methods for modulating the rate and/or extent of cell wall deposition in plant cells. In particular, methods for modulating the rate and/or extent of secondary cell wall deposition, including cell wall lignification, would be particularly desirable.

Reference to any prior art in this specification is not, and should not be taken as, an acknowledgment or any form of suggestion that this prior art forms part of the common general knowledge in any country.

### SUMMARY OF THE INVENTION

The present invention is predicated, in part, on the functional characterisation of a homeodomain/leucine zipper (HD-Zip) polypeptide. In a specific embodiment of the invention, it has been determined that expression of a HD-Zip polypeptide, particularly a HD-Zip II, effects repression or inhibition of various aspects of cell wall deposition, particularly secondary cell wall deposition, in plant cells.

In a first aspect, the present invention provides a method for modulating the rate and/or extent of cell wall deposition in a plant cell, the method comprising modulating the expression of a homeodomain/leucine zipper (HD-Zip) polypeptide in the plant cell.

In some embodiments, reference herein to “cell wall deposition” should be understood to refer to secondary cell wall deposition.

In some specific embodiments, the HD-Zip polypeptide is a class II HD-Zip polypeptide (HD-Zip II polypeptide).

As referred to herein, the “rate and/or extent of cell wall deposition” should be understood to include, but not be limited to, the actual rate and/or extent of cell wall deposition by a plant cell. The “rate and/or extent of cell wall deposition” should also be understood to include any process in the plant cell which is involved in or associated with cell wall deposition.

In one embodiment, modulating the rate and/or extent of cell wall deposition in the plant cell comprises modulating the expression of one or more secondary cell wall biosynthetic enzymes in the plant cell. Thus, also provided is a method for modulating the expression of one or more secondary cell wall biosynthetic enzymes in a plant cell, the method comprising modulating the expression of a homeodomain/leucine zipper (HD-Zip) polypeptide in the plant cell.

In another embodiment, modulating the rate and/or extent of cell wall deposition in the plant cell comprises modulating the rate and/or extent of lignin deposition in the cell wall of the plant cell. Thus, also provided is a method for modulating the rate and/or extent of lignin deposition in the cell wall of a plant cell, the method comprising modulating the expression of a homeodomain/leucine zipper (HD-Zip) polypeptide in the plant cell.

As set out in detail later, it has been determined that expression of an HD-Zip polypeptide also has an effect on cell size and morphology. Thus, in further embodiments, modulating the rate and/or extent of cell wall deposition further effects a modulation in the size of the plant cell as measured in at least one dimension.

In a second aspect, the present invention provides a genetically modified plant cell comprising a modulated rate and/or extent of cell wall deposition relative to an unmodified form of the cell, wherein modulation of the rate and/or extent of cell wall deposition is effected by modulation of the expression of a homeodomain/leucine zipper (HD-Zip) polypeptide in the genetically modified cell, relative to an unmodified form of the cell.

In one particular embodiment, the cell comprises a modulated rate and/or extent of secondary cell wall deposition.

As set out with respect to the first aspect of the invention, the modulated rate and/or extent of cell wall deposition in the cell may include the actual rate and/or extent of cell wall deposition by a plant cell and/or any process in the plant cell which is involved in or associated with cell wall deposition.

In a further embodiment, the size of the plant cell, as measured in at least one dimension, may also be modulated.

In a third aspect, the present invention provides a plant or a part, organ or tissue thereof comprising one or more cells according to the second aspect of the invention.

In a fourth aspect, the present invention provides a plant cell culture or plant tissue culture comprising one or more cells according to the second aspect of the invention.

In a fifth aspect, the present invention provides a method determining and/or predicting the rate and/or extent of cell wall deposition in a plant, or a part, organ, tissue or cell thereof, the method comprising determining the expression of a homeodomain/leucine zipper (HD-Zip) polypeptide in the plant or a part, organ, tissue or cell thereof.

The method of the fifth aspect of the present invention may be used, among other things, to select a plant, or part, organ, tissue or cell thereof, which has a desired level of cell wall deposition.

In one particular embodiment, the method is for determining the rate and/or extent of secondary cell wall deposition in a plant, or a part, organ, tissue or cell thereof.

In the method of the fifth aspect of the invention, the rate and/or extent of cell wall deposition determined and/or predicted in accordance with the method may include the actual rate and/or extent of cell wall deposition by a plant cell and/or the rate and/or extent of any process in the plant cell which is involved in or associated with cell wall deposition.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Nucleotide and amino acid sequences are referred to herein by a sequence identifier number (SEQ ID NO:). A summary of the sequence identifiers is provided in Table 1. A sequence listing is provided at the end of the specification.

TABLE 1
Summary of Sequence Identifiers

| | | Sequence |
|---|---|---|
| Sequence | | Listing |
| Identifier | Sequence | Entry |
| SEQ ID NO: 1 | TaHD-Zip II-1 amino acid sequence | 400 <1> |
| SEQ ID NO: 2 | TaHD-Zip II-1 cDNA nucleotide sequence | 400 <2> |
| SEQ ID NO: 3 | TaHDZipII-1 forward primer | 400 <3> |
| SEQ ID NO: 4 | TaHDZipII-1 reverse primer | 400 <4> |
| SEQ ID NO: 5 | Laccase 1 forward primer | 400 <5> |
| SEQ ID NO: 6 | Laccase 1 reverse primer | 400 <6> |
| SEQ ID NO: 7 | Laccase 2 forward primer | 400 <7> |
| SEQ ID NO: 8 | Laccase 2 reverse primer | 400 <8> |
| SEQ ID NO: 9 | HvCesA4 forward primer | 400 <9> |
| SEQ ID NO: 10 | HvCesA4 reverse primer | 400 <10> |
| SEQ ID NO: 11 | HvCesA7 forward primer | 400 <11> |
| SEQ ID NO: 12 | HvCesA7 reverse primer | 400 <12> |
| SEQ ID NO: 13 | HvCesA8 forward primer | 400 <13> |
| SEQ ID NO: 14 | HvCesA8 reverse primer | 400 <14> |
| SEQ ID NO: 15 | HvCesA1 forward primer | 400 <15> |
| SEQ ID NO: 16 | HvCesA1 reverse primer | 400 <16> |
| SEQ ID NO: 17 | HvCesA3 forward primer | 400 <17> |
| SEQ ID NO: 18 | HvCesA3 reverse primer | 400 <18> |
| SEQ ID NO: 19 | HvCesA7 partial cDNA nucleotide | 400 <19> |
| | sequence | |

### DESCRIPTION OF EXEMPLARY EMBODIMENTS

It is to be understood that the following description is for the purpose of describing particular embodiments only and is not intended to be limiting with respect to the above description.

In a first aspect, the present invention provides a method for modulating the rate and/or extent of cell wall deposition in a plant cell, the method comprising modulating the expression of a homeodomain/leucine zipper (HD-Zip) polypeptide in the plant cell.

Plant cells are typically enclosed by a cell wall containing cellulose. The cell wall has a number of functions: it lends the cell stability, it determines its shape, influences its development, protects the cell against pathogens and counterbalances osmotic pressure. The cell wall of elongating cells is elastic, a property which is generally lost in fully differentiated cells.

Cell walls can be classified as primary or secondary walls. The primary cell wall is laid out during the first division of the cell. It develops normally between the two daughter cells during early telophase.

The early stage of the new cell wall is the cell plate, a lamella-like structure in the former equatorial plane of the mitotic apparatus. Electron microscopic studies show that it develops by fusion of numerous vesicles. The plate grows centrifugally until it reaches the longitudinal lateral walls of the mother cell. Electron dense material is deposited at both its sides. The thus developing structure is called the phragmoplast. It is the immediate precursor of the primary wall.

Primary cell walls are generally deposited during cell wall expansion or elongation and are composed mostly of polysaccharides (approx 90%) such as cellulose, pectin, heteroxylans, xyloglucans, 1-3,1-4-β glucans, mannans. Primary cell walls may also contain approximately 5-10% proteins including both structural and enzymatic proteins. Primary cell walls may also contain phenolic compounds.

As set out above, the disclosed method contemplates modulating the rate and/or extent of cell wall deposition in a plant cell. As such, this may include modulating the rate and/or extent of primary and/or secondary cell wall deposition in a plant cell.

However, in some embodiments, reference herein to “cell wall deposition” should be understood to refer to secondary cell wall deposition. Thus, in some embodiments, the disclosed method is for modulating the rate and/or extent of secondary cell wall deposition in a plant cell.

The term “secondary cell wall”, as used herein, generally refers to cell wall material which is deposited after cessation of cell wall expansion.

The secondary wall develops by successive encrustation and deposition of cellulose fibrils and other components on the inside of the primary cell wall. Secondary cell wall deposition generally occurs when the cell has stopped growth and wall elasticity is no longer required. While the primary wall structure is generally similar across plant cell types and species, there are cell type and species-specific differences typical for the secondary cell wall.

The most striking feature of secondary walls is their loss of plasticity. Progressive depositions of new lamellas thicken the wall while the cell lumen's diameter decreases.

Secondary cell walls are generally less hydrated than primary walls and contain less pectins and hemicellulose. Instead other components are deposited, which are sometimes characteristic for certain cell groups or tissues.

Secondary cell walls can also be lignified. Lignin is the basic unit of xylem- and strengthening elements (wood) and consists of polymerized phenylpropane units. The three most important starting compounds are coumaryl alcohol (with an OH-group in position 4 of the phenyl ring), coniferyl alcohol (OH-group in position 4, —OCH₃ in position 3) and sinapyl alcohol (OH-group in position 4, —OCH₃ group in positions 3 and 5).

The lignins of plant groups differ in the percentages of these starting compounds and in the way they are linked. All bonds leading to the formation of a three-dimensional molecular network are covalent. As a consequence lignins form a network that provides stability. However, the bonds are irreversible, and stretching of the wall and growth of the cell are generally impossible after substantial wall lignification.

The lignin of pteridophytes consists mainly of coniferyl alcohol polymers, while in dicots coniferyl and sinapyl alcohol polymers occur in roughly equal amounts. In the lignins of all plant groups are only trace amounts of coumaryl alcohol are found.

Mannans may also be incorporated into secondary walls, and are a structural element of many seeds. The secondary walls of pollen also contain sporopollenin, a polymerization product of carotene.

Many secondary walls also contain a wide range of strongly hydrophobic compounds, like suberine, the basic component of cork. Such compounds may comprise integral components of the wall itself. Alternatively, such compounds may be deposited on the wall as solid excretion products (cuticle, wax deposits, etc.).

Beside the structural elements of the wall, non-structural components may also be part of the secondary cell wall. These components may include a number of low molecular weight compounds (dyes, alcohols, terpenes, tannins, etc.), oligosaccharides (and polysaccharides) of different configurations as well as proteins (usually glycoproteins). Some of them participate in recognition processes, such as incompatibility factors at the stigma surface and several carbohydrate-binding lectins.

As set out above, the present invention is predicated, in part, on modulating the rate and/or extent of cell wall deposition in a plant cell.

As referred to herein, “modulation” of the rate and/or extent of cell wall deposition in a plant cell should be understood to include an increase or decrease in the rate and/or extent of cell wall deposition in a plant cell.

By “increasing” is intended, for example, a 1%, 5%, 10%, 20%, 30%, 40%, 50%, 60%, 70%, 80%, 90%, 100%, 2-fold, 5-fold, 10-fold, 20 fold, 50-fold, 100-fold increase in the rate and/or extent of cell wall deposition in the plant cell. By “decreasing” is intended, for example, a 1%, 5%, 10%, 20%, 30%, 40%, 50%, 60%, 70%, 80%, 90%, 91%, 92%, 93%, 94%, 95%, 96%, 97%, 98%, 99%, 100% reduction in the rate and/or extent of cell wall deposition in the plant cell.

“Modulating” should also be understood to include introducing cell wall deposition into a plant cell which does not have a primary and/or secondary cell wall, such as protoplasts, some algae and the like. In addition, “modulating” may also include the substantially complete inhibition of primary and/or secondary cell wall deposition in a plant cell.

As set out above, the disclosed method contemplates modulating the expression of a “homeodomain/leucine zipper (HD-Zip) polypeptide” in the plant cell.

“HD-Zip polypeptides” include polypeptides that comprise both homeodomain and leucine zipper structural motifs.

The homeodomain motif is a protein structural domain that binds DNA and is thus commonly found in transcription factors. The fold consists of a 60-amino acid helix-turn-helix structure in which three alpha helices are connected by short loop regions. The N-terminal two helices are antiparallel and the longer C-terminal helix is roughly perpendicular to the axes established by the first two.

Genetic and structural analyses of the homeodomain suggest a general model for homeodomain binding to DNA, in which the most highly conserved of three α-helices (helix 3) fits directly into the major groove of DNA.

Homeodomains can bind both specifically and nonspecifically to B-DNA with the C-terminal recognition helix aligning in the DNA's major groove and the unstructured peptide “tail” at the N-terminus aligning in the minor groove. The recognition helix and the inter-helix loops are rich in arginine and lysine residues, which form hydrogen bonds to the DNA backbone; conserved hydrophobic residues in the center of the recognition helix aid in stabilizing the helix packing. Homeodomain proteins show a preference for the DNA sequence 5′-ATTA-3′; while sequence-independent binding occurs with significantly lower affinity.

HD-Zip polypeptides also comprise a leucine zipper structural motif in addition to a homeodomain structural motif.

The main feature of the leucine zipper motif is the predominance of the common amino acid leucine at the d position of a heptad repeat. Leucine zippers were first identified by sequence alignment of certain transcription factors which identified a common pattern of leucines every seven amino acids. These leucines were later shown to form the hydrophobic core of a coiled coil. Each half of a leucine zipper consists of a short alpha-helix with a leucine residue at every seventh position. The standard 3.6 residues per turn alpha-helix structure changes slightly to become a 3.5 residues per turn alpha-helix. In this structure, one leucine comes in direct contact with another leucine on the other strand every second turn.

HD-Zip polypeptides are known to function as mediators of plant development. In some cases, these polypeptides couple a developmental response to an environmental signal. For example, developmental responses such as the shade avoidance response and drought response are regulated by HD-Zip polypeptides.

HD-Zip polypeptides may be classified into the HD-Zip I, HD-Zip II, HD-Zip III, and HD-Zip IV subfamilies. For details of the classification of HD-Zip polypeptides into the various subfamilies, see Meijer et al. (Plant J. 11: 263-276, 1997) and Aso et al. (Mol. Biol. Evol. 16: 544-551, 1999).

HD-Zip I and II genes have been demonstrated to be involved in the signal transduction networks of light, dehydration-induced ABA and auxin. These signal transduction networks are related to the general growth regulation of plants. Members of the HD-Zip III subfamily have been shown to play roles in cell differentiation in the stele, although the functions of some genes remain unknown.

HD-Zip IV genes have been shown to be related to the differentiation of the outermost cell layer.

In some specific embodiments described herein, the HD-Zip polypeptide is a class II HD-Zip polypeptide (HD-Zip II polypeptide).

In further specific embodiments, the HD-Zip polypeptide comprises the amino acid sequence set forth in SEQ ID NO: 1 or an amino acid sequence which is at least 50% identical thereto.

The HD-Zip polypeptide having the defined level of sequence identity with SEQ ID NO: 1 may be a polypeptide which has one or more amino acid insertions, deletions or substitutions relative to the polypeptide comprising the amino acid sequence set forth in SEQ ID NO: 1; a mutant form or allelic variant of the polypeptide comprising the amino acid sequence set forth in SEQ ID NO: 1; an ortholog of the polypeptide comprising the amino acid sequence set forth in SEQ ID NO: 1; an analog of the polypeptide comprising the amino acid sequence set forth in SEQ ID NO: 1; and the like.

In some embodiments, reference herein to “at least 50%” sequence identity with regard to SEQ ID NO: 1, should be understood to encompass higher levels of sequence identity, including at least 60% amino acid sequence identity, at least 70% amino acid sequence identity, at least 80% amino acid sequence identity, at least 85% amino acid sequence identity, at least 90% amino acid sequence identity or at least 95%, 96%, 97%, 98%, 99% or 100% amino acid sequence identity to SEQ ID NO: 1.

When comparing amino acid sequences, the compared sequences should be compared over a comparison window of at least 50 amino acid residues, at least 100 amino acid residues, at least 200 amino acid residues, at least 250 amino acid residues or over the full length of SEQ ID NO: 1. The comparison window may comprise additions or deletions (ie. gaps) of about 20% or less as compared to the reference sequence (which does not comprise additions or deletions) for optimal alignment of the two sequences. Optimal alignment of sequences for aligning a comparison window may be conducted by computerized implementations of algorithms such the BLAST family of programs as, for example, disclosed by Altschul et al. (Nucl. Acids Res. 25: 3389-3402, 1997). A detailed discussion of sequence analysis can be found in Unit 19. 3 of Ausubel et al. (“Current Protocols in Molecular Biology” John Wiley & Sons Inc, 1994-1998, Chapter 15, 1998).

The present invention contemplates any means by which the level and/or activity of a HD-Zip polypeptide in a cell may be modulated. This includes, for example, methods such as the application of agents which modulate HD-Zip polypeptide activity in a cell, including the application of a HD-Zip polypeptide agonist or antagonist; the application of agents which mimic HD-Zip polypeptide activity in a cell; modulating the expression of a HD-Zip polypeptide encoding nucleic acid in the cell; or effecting the expression of an altered or mutated HD-Zip polypeptide encoding nucleic acid in a cell such that a HD-Zip polypeptide with increased or decreased specific activity, half-life and/or stability is expressed by the cell.

In one embodiment, the level and/or activity of the HD-Zip polypeptide is modulated by modulating the expression of a HD-Zip polypeptide encoding nucleic acid in the cell.

The term “modulating” with regard to the expression of a HD-Zip polypeptide encoding nucleic acid may include increasing or decreasing the transcription and/or translation of a HD-Zip polypeptide encoding nucleic acid in the cell. By “increasing” is intended, for example a 1%, 5%, 10%, 20%, 30%, 40%, 50%, 60%, 70%, 80%, 90%, 100%, 2-fold, 5-fold, 10-fold, 20-fold, 50-fold, 100-fold or greater increase in the transcription and/or translation of a HD-Zip polypeptide encoding nucleic acid. By “decreasing” is intended, for example, a 1%, 5%, 10%, 20%, 30%, 40%, 50%, 60%, 70%, 80%, 90%, 91%, 92%, 93%, 94%, 95%, 96%, 97%, 98%, 99%, 100% reduction in the transcription and/or translation of a HD-Zip polypeptide encoding nucleic acid. Modulating also comprises introducing expression of a HD-Zip polypeptide encoding nucleic acid not normally found in a particular cell; or the substantially complete inhibition (eg. knockout) of expression of a HD-Zip polypeptide encoding nucleic acid in a cell that normally has such activity.

As referred to herein, an “HD-Zip polypeptide encoding nucleic acid” refers to any nucleic acid which encodes an HD-Zip polypeptide, as hereinbefore defined.

In some embodiments, the HD-Zip polypeptide-encoding nucleic acid comprises the nucleotide sequence set forth in SEQ ID NO: 2 or a nucleotide sequence which is at least 50% identical thereto.

The HD-Zip polypeptide-encoding nucleic acid having the defined level of sequence identity with SEQ ID NO: 2 may be a nucleic acid which has one or more nucleotide insertions, deletions or substitutions relative to the nucleic acid comprising the nucleotide sequence set forth in SEQ ID NO: 2; a mutant form or allelic variant of the nucleotide sequence set forth in SEQ ID NO: 2; an ortholog of the nucleotide sequence set forth in SEQ ID NO: 2; and the like.

Reference herein to “at least 50%” sequence identity with regard to SEQ ID NO: 2, in some embodiments at least to encompass higher levels of sequence identity, including at least 60% amino acid sequence identity, at least 70% amino acid sequence identity, at least 80% amino acid sequence identity, at least 85% amino acid sequence identity, at least 90% amino acid sequence identity or at least 95%, 96%, 97%, 98%, 99% or 100% amino acid sequence identity to SEQ ID NO: 1.

When comparing nucleotide sequences, the compared sequences should be compared over a comparison window of at least 100 nucleotide residues, at least 200 nucleotide residues, at least 300 nucleotide residues, at least 400 nucleotide residues, at least 500 nucleotide residues, at least 600 nucleotide residues, at least 800 nucleotide residues, at least 1000 nucleotide residues, or over the full length of SEQ ID NO: 2. The comparison window may comprise additions or deletions (ie. gaps) of about 20% or less as compared to the reference sequence (which does not comprise additions or deletions) for optimal alignment of the two sequences. Optimal alignment of sequences for aligning a comparison window may be conducted by computerized implementations of algorithms such the BLAST family of programs as, for example, disclosed by Altschul et al. (Nucl. Acids Res. 25: 3389-3402, 1997). A detailed discussion of sequence analysis can be found in Unit 19. 3 of Ausubel et al. (“Current Protocols in Molecular Biology” John Wiley & Sons Inc, 1994-1998, Chapter 15, 1998).

The present invention contemplates any means by which the expression of a HD-Zip polypeptide encoding nucleic acid may be modulated. For example, exemplary methods for modulating the expression of a HD-Zip polypeptide encoding nucleic acid include, for example: genetic modification of the cell to upregulate or downregulate expression of an endogenous HD-Zip polypeptide encoding nucleic acid; genetic modification by transformation with a HD-Zip polypeptide encoding nucleic acid; genetic modification to increase the copy number of a HD-Zip polypeptide encoding nucleic acid sequence in the cell; administration of a nucleic acid molecule to the cell which modulates expression of an endogenous HD-Zip polypeptide encoding nucleic acid in the cell; and the like.

In one embodiment, the expression of a HD-Zip polypeptide encoding nucleic acid is modulated by genetic modification of the cell. The term “genetically modified”, as used herein, should be understood to include any genetic modification that effects an alteration in the expression of a HD-Zip polypeptide encoding nucleic acid in the genetically modified cell relative to a non-genetically modified form of the cell. Exemplary types of genetic modification include: random mutagenesis such as transposon, chemical, UV and phage mutagenesis together with selection of mutants which overexpress or underexpress an endogenous HD-Zip polypeptide encoding nucleic acid; transient or stable introduction of one or more nucleic acid molecules into a cell which direct the expression and/or overexpression of HD-Zip polypeptide encoding nucleic acid in the cell; inhibition of an endogenous HD-Zip polypeptide encoding nucleic acid by site-directed mutagenesis of an endogenous HD-Zip polypeptide encoding nucleic acid; introduction of one or more nucleic acid molecules which inhibit the expression of an endogenous HD-Zip polypeptide encoding nucleic acid in the cell, eg. a cosuppression construct or an RNAi construct; and the like.

In one specific embodiment, the present invention contemplates increasing the level of HD-Zip polypeptide in a cell, by introducing the expression of a HD-Zip polypeptide encoding nucleic acid into the cell, upregulating the expression of a HD-Zip polypeptide encoding nucleic acid in the cell and/or increasing the copy number of a HD-Zip polypeptide encoding nucleic acid in the cell. In one embodiment, the introduced HD-Zip polypeptide encoding nucleic acid may be placed under the control of a transcriptional control sequence such as a native promoter or a heterologous promoter.

In these embodiments, an increase in the expression of the HD-Zip polypeptide in the plant cell effects a decrease in the rate and/or extent of secondary cell wall deposition in a plant cell.

Methods for plant transformation and expression of an introduced nucleotide sequence are well known in the art, and the present invention contemplates the use of any suitable method.

Suitable methods for the transformation of plant cells include, for example: Agrobacterium-mediated transformation, microprojectile bombardment based transformation methods and direct DNA uptake based methods. Roa-Rodriguez et al. (Agrobacterium-mediated transformation of plants, 3rd Ed. CAMBIA Intellectual Property Resource, Can berra, Australia, 2003) review a wide array of suitable Agrobacterium-mediated plant transformation methods for a wide range of plant species. Bacterial mediated transformation using bacteria other than Agrobacterium sp. may also be used, for example as described in Broothaerts et al. (Nature 433: 629-633, 2005). Microprojectile bombardment may also be used to transform plant tissue and methods for the transformation of plants, particularly cereal plants, and such methods are reviewed by Casas et al. (Plant Breeding Rev. 13: 235-264, 1995). Direct DNA uptake transformation protocols such as protoplast transformation and electroporation are described in detail in Galbraith et al. (eds.), Methods in Cell Biology Vol. 50, Academic Press, San Diego, 1995). In addition to the methods mentioned above, a range of other transformation protocols may also be used. These include infiltration, electroporation of cells and tissues, electroporation of embryos, microinjection, pollen-tube pathway, silicon carbide- and liposome mediated transformation. Methods such as these are reviewed by Rakoczy-Trojanowska (Cell. Mol. Biol. Lett. 7: 849-858, 2002). A range of other plant transformation methods may also be evident to those of skill in the art. By way of further example, reference is also made to Zhao et al. (Mol Breeding DOI 10.1007/s11032-006-9005-6, 2006), Katsuhara et al. (Plant Cell Physiol 44(12): 1378-1383, 2003), Ohta et al. (FEBS Letters 532: 279-282, 2002) and Wu et al. (Plant Science 169: 65-73, 2005).

In further embodiments the present invention also provides methods for down-regulating expression of a HD-Zip polypeptide encoding nucleic acid in a cell. In these embodiments, a decrease in the expression of the HD-Zip polypeptide in the plant cell effects an increase in the rate and/or extent of secondary cell wall deposition in a plant cell.

The present invention contemplates methods such as knockout or knockdown of an endogenous HD-Zip polypeptide encoding nucleic acid in a cell using methods including, for example:
(i) insertional mutagenesis of a HD-Zip polypeptide encoding nucleic acid in a cell including knockout or knockdown of a HD-Zip polypeptide encoding nucleic acid in a cell by homologous recombination with a knockout construct (for an example of targeted gene disruption in plants see Terada et al., Nat. Biotechnol. 20: 1030-1034, 2002);
(ii) post-transcriptional gene silencing (PTGS) or RNAi of a HD-Zip polypeptide encoding nucleic acid in a cell (for review of PTGS and RNAi see Sharp, Genes Dev. 15(5): 485-490, 2001; and Hannon, Nature 418: 244-51, 2002);
(iii) transformation of a cell with an antisense construct directed against a HD-Zip polypeptide encoding nucleic acid (for examples of antisense suppression in plants see van der Krol et al., Nature 333: 866-869; van der Krol et al., BioTechniques 6: 958-967; and van der Krol et al., Gen. Genet. 220: 204-212);
(iv) transformation of a cell with a co-suppression construct directed against a HD-Zip polypeptide encoding nucleic acid (for an example of co-suppression in plants see van der Krol et al., Plant Cell 2(4): 291-299);
(v) transformation of a cell with a construct encoding a double stranded RNA directed against a HD-Zip polypeptide encoding nucleic acid (for an example of dsRNA mediated gene silencing see Waterhouse et al., Proc. Natl. Acad. Sci. USA 95: 13959-13964, 1998);
(vi) transformation of a cell with a construct encoding an siRNA or hairpin RNA directed against a HD-Zip polypeptide encoding nucleic acid (for an example of siRNA or hairpin RNA mediated gene silencing in plants see Lu et al., Nucl. Acids Res. 32(21): e171; doi:10.1093/nar/gnh170, 2004); and
(vii) insertion of a miRNA target sequence such that it is in operable connection with an HD-Zip polypeptide encoding nucleic acid (for an example of miRNA mediated gene silencing see Brown et al., Blood 110(13): 4144-4152, 2007).

The present invention also facilitates the downregulation of a HD-Zip polypeptide encoding nucleic acid in a cell via the use of synthetic oligonucleotides, for example, siRNAs or microRNAs directed against a HD-Zip polypeptide encoding nucleic acid (for examples of synthetic siRNA mediated silencing see Caplen et al., Proc. Natl. Acad. Sci. USA 98: 9742-9747, 2001; Elbashir et al., Genes Dev. 15: 188-200, 2001; Elbashir et al., Nature 411: 494-498, 2001; Elbashir et al., EMBO J. 20: 6877-6888, 2001; and Elbashir et al., Methods 26: 199-213, 2002).

In addition to the examples above, the introduced nucleic acid may also comprise a nucleotide sequence which is not directly related to a HD-Zip polypeptide encoding nucleic acid but, nonetheless, may directly or indirectly modulate the expression of a HD-Zip polypeptide encoding nucleic acid in a cell. Examples include nucleic acid molecules that encode transcription factors or other proteins which promote or suppress the expression of an endogenous HD-Zip polypeptide encoding nucleic acid in a cell; and other non-translated RNAs (such as an miRNA) which directly or indirectly promote or suppress endogenous HD-Zip polypeptide encoding nucleic acid expression and the like.

In order to effect expression of an introduced nucleic acid in a genetically modified cell, where appropriate, the introduced nucleic acid may be operably connected to one or more transcriptional control sequences and/or promoters.

For the purposes of the present specification, a transcriptional control sequence is regarded as “operably connected” to a given gene or other nucleotide sequence when the transcriptional control sequence is able to promote, inhibit or otherwise modulate the transcription of the gene or other nucleotide sequence.

A promoter may regulate the expression of an operably connected nucleotide sequence constitutively, or differentially, with respect to the cell, tissue, organ or developmental stage at which expression occurs, in response to external stimuli such as physiological stresses, pathogens, or metal ions, amongst others, or in response to one or more transcriptional activators. As such, the promoter used in accordance with the methods of the present invention may include, for example, a constitutive promoter, an inducible promoter, a tissue-specific promoter or an activatable promoter.

Plant constitutive promoters typically direct expression in nearly all tissues of a plant and are largely independent of environmental and developmental factors. Examples of constitutive promoters that may be used in accordance with the present invention include plant viral derived promoters such as the Cauliflower Mosaic Virus 35S and 19S (CaMV 35S and CaMV 19S) promoters; bacterial plant pathogen derived promoters such as opine promoters derived from Agrobacterium spp., eg. the Agrobacterium-derived nopaline synthase (nos) promoter; and plant-derived promoters such as the rubisco small subunit gene (rbcS) promoter, the plant ubiquitin promoter (Pubi) and the rice actin promoter (Pact).

“Inducible” promoters include, but are not limited to, chemically inducible promoters and physically inducible promoters. Chemically inducible promoters include promoters which have activity that is regulated by chemical compounds such as alcohols, antibiotics, steroids, metal ions or other compounds. Examples of chemically inducible promoters include: alcohol regulated promoters (eg. see European Patent 637 339); tetracycline regulated promoters (eg. see U.S. Pat. No. 5,851,796 and U.S. Pat. No. 5,464,758); steroid responsive promoters such as glucocorticoid receptor promoters (eg. see U.S. Pat. No. 5,512,483), estrogen receptor promoters (eg. see European Patent Application 1 232 273), ecdysone receptor promoters (eg. see U.S. Pat. No. 6,379,945) and the like; metal-responsive promoters such as metallothionein promoters (eg. see U.S. Pat. No. 4,940,661, U.S. Pat. No. 4,579,821 and U.S. Pat. No. 4,601,978); and pathogenesis related promoters such as chitinase or lysozyme promoters (eg. see U.S. Pat. No. 5,654,414) or PR protein promoters (eg. see U.S. Pat. No. 5,689,044, U.S. Pat. No. 5,789,214, Australian Patent 708850, U.S. Pat. No. 6,429,362).

The inducible promoter may also be a physically regulated promoter which is regulated by non-chemical environmental factors such as temperature (both heat and cold), light and the like. Examples of physically regulated promoters include heat shock promoters (eg. see U.S. Pat. No. 5,447,858, Australian Patent 732872, Canadian Patent Application 1324097); cold inducible promoters (eg. see U.S. Pat. No. 6,479,260, U.S. Pat. No. 6,184,443 and U.S. Pat. No. 5,847,102); light inducible promoters (eg. see U.S. Pat. No. 5,750,385 and Canadian Patent 132 1563); light repressible promoters (eg. see New Zealand Patent 508103 and U.S. Pat. No. 5,639,952).

“Tissue specific promoters” include promoters which are preferentially or specifically expressed in one or more specific cells, tissues or organs in an organism and/or one or more developmental stages of the organism. It should be understood that a tissue specific promoter may also be inducible.

Examples of plant tissue specific promoters include: root specific promoters such as those described in US Patent Application 2001047525; fruit specific promoters including ovary specific and receptacle tissue specific promoters such as those described in European Patent 316 441, U.S. Pat. No. 5,753,475 and European Patent Application 973 922; and seed specific promoters such as those described in Australian Patent 612326 and European Patent application 0 781 849 and Australian Patent 746032.

The promoter may also be a promoter that is activatable by one or more transcriptional activators, referred to herein as an “activatable promoter”. For example, the activatable promoter may comprise a minimal promoter operably connected to an Upstream Activating Sequence (UAS), which comprises, inter alia, a DNA binding site for one or more transcriptional activators.

As referred to herein the term “minimal promoter” should be understood to include any promoter that incorporates at least an RNA polymerase binding site and, optionally a TATA box and transcription initiation site and/or one or more CAAT boxes. In one embodiment wherein the cell is a plant cell, the minimal promoter may be derived from the Cauliflower Mosaic Virus 35S (CaMV 35S) promoter. The CaMV 35S derived minimal promoter may comprise, for example, a sequence that substantially corresponds to positions −90 to +1 (the transcription initiation site) of the CaMV 35S promoter (also referred to as a −90 CaMV 35S minimal promoter), −60 to +1 of the CaMV 35S promoter (also referred to as a −60 CaMV 35S minimal promoter) or −45 to +1 of the CaMV 35S promoter (also referred to as a −45 CaMV 35S minimal promoter).

As set out above, the activatable promoter may comprise a minimal promoter fused to an Upstream Activating Sequence (UAS). The UAS may be any sequence that can bind a transcriptional activator to activate the minimal promoter. Exemplary transcriptional activators include, for example: yeast derived transcription activators such as Gal4, Pdr1, Gcn4 and Ace1; the viral derived transcription activator, VP16; Hap1 (Hach et al., J Biol Chem 278: 248-254, 2000); Gaf1 (Hoe et al., Gene 215(2): 319-328, 1998); E2F (Albani et al., J Biol Chem 275: 19258-19267, 2000); HAND2 (Dai and Cserjesi, J Biol Chem 277: 12604-12612, 2002); NRF-1 and EWG (Herzig et al., J Cell Sci 113: 4263-4273, 2000); P/CAF (Itoh et al., Nucl Acids Res 28: 4291-4298, 2000); MafA (Kataoka et al., J Biol Chem 277: 49903-49910, 2002); human activating transcription factor 4 (Liang and Hai, J Biol Chem 272: 24088-24095, 1997); Bcl10 (Liu et al., Biochem Biophys Res Comm 320(1): 1-6, 2004); CREB-H (Omori et al., Nucl Acids Res 29: 2154-2162, 2001); ARR1 and ARR2 (Sakai et al., Plant J 24(6): 703-711, 2000); Fos (Szuts and Bienz, Proc Natl Acad Sci USA 97: 5351-5356, 2000); HSF4 (Tanabe et al., J Biol Chem 274: 27845-27856, 1999); MAML1 (Wu et al., Nat Genet 26: 484-489, 2000).

In one embodiment, the UAS comprises a nucleotide sequence that is able to bind to at least the DNA-binding domain of the GAL4 transcriptional activator. UAS sequences, which can bind transcriptional activators that comprise at least the GAL4 DNA binding domain, are referred to herein as UASG. In another embodiment, the UASG comprises the sequence 5′-CGGAGTACTGTCCTCCGAG-3′ or a functional homolog thereof.

As referred to herein, a “functional homolog” of the UASG sequence should be understood to refer to any nucleotide sequence which can bind at least the GAL4 DNA binding domain and which may comprise a nucleotide sequence having at least 50% identity, at least 65% identity, at least 80% identity or at least 90% identity with the UASG nucleotide sequence.

The UAS sequence in the activatable promoter may comprise a plurality of tandem repeats of a DNA binding domain target sequence. For example, in its native state, UASG comprises four tandem repeats of the DNA binding domain target sequence. As such, the term “plurality” as used herein with regard to the number of tandem repeats of a DNA binding domain target sequence should be understood to include, for example, at least 2 tandem repeats, at least 3 tandem repeats or at least 4 tandem repeats.

The transcriptional control sequence to which the HD-Zip encoding nucleic acid is connected may be introduced into the cell with the HD-Zip encoding nucleic acid itself, or alternatively, the HD-Zip encoding nucleic acid may be inserted into the genome of the plant cell such that it becomes operably connected to an endogenous transcriptional control sequence. In the latter embodiments, the insertion of the HD-Zip encoding nucleic acid in the genome such that it is under the control of an endogenous transcriptional control sequence may be the result of either non-site directed or random DNA insertion or the result of site-directed insertion (for example as described in (Terada et al., Nat. Biotechnol. 20: 1030-1034, 2002).

As set out above, the disclosed method provides a method for modulating the rate and/or extent of cell wall deposition in a plant cell. As referred to herein, the “rate and/or extent of cell wall deposition” should be understood to include, but not be limited to the actual rate and/or extent of cell wall deposition by a plant cell. The “rate and/or extent of cell wall deposition” should also be understood to include any process in the plant cell which is involved in or associated with cell wall deposition. For example, and as discussed later, modulation of the rate and/or extent of cell wall deposition may include any one or more of: modulation of actual cell wall deposition, modulation of the expression of cell wall biosynthetic enzymes, modulation of the amount of one or more primary or secondary cell wall components in the plant cell wall, and the like.

Furthermore, modulation of the rate or extent of cell wall deposition should be understood to include, for example, modulation of the rate and/or extent of cell wall production or degradation in the plant cell.

In one embodiment, modulating the rate and/or extent of cell wall deposition in the plant cell comprises modulating the expression of one or more secondary cell wall biosynthetic enzymes in the plant cell.

As referred to herein, “modulating the expression of one or more secondary cell wall biosynthetic enzymes in the plant cell” refers to the upregulation or downregulation of one or more biosynthetic enzymes involved in the deposition of secondary cell wall in a plant cell.

Thus, also provided is a method for modulating the expression of one or more secondary cell wall biosynthetic enzymes in a plant cell, the method comprising modulating the expression of a homeodomain/leucine zipper (HD-Zip) polypeptide in the plant cell.

As referred to herein, the “modulating the expression of one or more secondary cell wall biosynthetic enzymes” should be understood to include any process that effects the level and/or activity of one or more secondary cell wall biosynthetic enzymes in a plant cell. In one particular embodiment, however, this term should be understood to encompass modulation of the transcription and/or translation of a nucleic acid which encodes a secondary cell wall biosynthetic enzyme.

Exemplary secondary cell wall biosynthetic enzymes include, for example, cellulose synthases, xylan synthases and other polysaccharide synthases, peroxidases and laccases.

In one embodiment an increase in the expression of the HD-Zip polypeptide in the plant cell effects a decrease in the expression of one or more secondary cell wall biosynthetic enzymes. In an alternate embodiment, a decrease in the expression of the HD-Zip polypeptide in the plant cell effects an increase in the expression of one or more secondary cell wall biosynthetic enzymes.

In one specific embodiment, the one or more secondary cell wall biosynthetic enzymes comprise a laccase. In a further specific embodiment the one or more secondary cell wall biosynthetic enzymes comprise a laccase 1.

In another embodiment, the one or more secondary cell wall biosynthetic enzymes comprise a cellulose synthase or cellulose synthase like enzyme.

Cellulose synthases may be encoded by cellulose synthase (CesA) genes, while cellulose synthase like enzymes may be encoded by the Csl gene family. Both the CesA genes and the cellulose synthase-like (Csl) gene family form a large gene superfamily.

The Csl gene superfamily, inclusive of the CesA gene family, is described in detail in the literature and will not be further discussed herein. In this regard, reference is made to Burton et al. (Plant Physiol. 134(1): 224-236, 2004), Richmond and Somerville (Plant Physiol. 124: 495-498, 2000) and Burton et al. (Science 311 (5769): 1940-2, 2006).

In some specific embodiments of the invention, the one or more secondary cell wall biosynthetic enzymes comprise a cellulose synthase selected from the list consisting of CesA4, CesA7 and CesA8.

In another embodiment, modulating the rate and/or extent of cell wall deposition in the plant cell comprises modulating the rate and/or extent of lignin deposition in the cell wall of the plant cell.

As referred to herein, “modulating the rate and/or extent of lignin deposition in the cell wall of the plant cell” refers to increasing or decreasing the rate and/or extent of lignin deposition in the cell wall of the plant cell.

Thus, the present invention provides a method for modulating the rate and/or extent of lignin deposition in the cell wall of a plant cell, the method comprising modulating the expression of a homeodomain/leucine zipper (HD-Zip) polypeptide in the plant cell.

In one embodiment an increase in the expression of the HD-Zip polypeptide in the plant cell effects a decrease in the rate and/or extent of lignin deposition in the cell wall of the plant cell. In an alternate embodiment, a decrease in the expression of the HD-Zip polypeptide in the plant cell effects an increase in the rate and/or extent of lignin deposition in the cell wall of the plant cell.

As set out in detail later, it has been determined that expression of an HD-Zip polypeptide also has an effect on cell size and morphology. Without limiting the present invention to any particular mode of action, it is postulated that increased expression of an HD-Zip polypeptide in a plant cell inhibits or delays secondary cell wall deposition such that the period of cell elongation can be lengthened. Furthermore, it is also considered that the opposite would also occur in that a decrease in, or inhibition of, the expression of an HD-Zip polypeptide in a cell would promote secondary cell wall deposition and thus constrain cell elongation.

Thus, in further embodiments, modulating the rate and/or extent of cell wall deposition further effects a modulation in the size of the plant cell as measured in at least one dimension.

In a yet further embodiment, an increase in the expression of the HD-Zip polypeptide in the plant cell effects an increase in the size of the plant cell as measured in at least one dimension.

As set out above, the disclosed method is practiced on a plant cell. As referred to herein a “plant cell” includes any cell from an organism of the kingdom Plantae. As such, the cell may be a bryophyte cell or a vascular plant cell. Generally, the cells used in accordance with the present invention include walled members of this kingdom. However, naturally non-walled members of the kingdom may be used and the present invention may be used to promote primary or secondary cell wall deposition in such cells.

In some embodiments, the cell is a monocotyledonous or dicotyledonous angiosperm plant cell or a gymnosperm plant cell. In one particular embodiment, the cell is a monocotyledonous plant cell, and in a further embodiment a cereal crop plant cell.

As used herein, the term “cereal crop plant” includes members of the Poales (grass family) that produce edible grain for human or animal food. Examples of Poales cereal crop plants which in no way limit the present invention include wheat, rice, maize, millets, sorghum, rye, triticale, oats, barley, teff, wild rice, spelt and the like. However, the term cereal crop plant should also be understood to include a number of non-Poales species that also produce edible grain and are known as the pseudocereals, such as amaranth, buckwheat and quinoa.

Although cereal crop plants are particularly suitable monocotyledonous plants, the other monocotyledonous plants may also be used, such as other non-cereal plants of the Poales, specifically including pasture grasses such as Lolium spp.

In a second aspect, the present invention provides a genetically modified plant cell comprising a modulated rate and/or extent of cell wall deposition relative to an unmodified form of the cell, wherein modulation of the rate and/or extent of cell wall deposition is effected by modulation of the expression of a homeodomain/leucine zipper (HD-Zip) polypeptide in the genetically modified cell, relative to an unmodified form of the cell.

As referred to herein, a “genetically modified cell” comprises a cell that is genetically modified with respect to the wild type of the cell. As such, a genetically modified cell may be a cell which has itself been genetically modified and/or the progeny of such a cell. As such, genetically modified cells include, for example, transgenic cells and mutant cells or the progeny of such cells which retain the genetic modification relative to a wild type cell.

As set out above, the plant cell of the second aspect of the invention comprises a modulated rate and/or extent of cell wall deposition relative to an unmodified form of the cell. This should be understood as a difference in the rate and/or extent of primary and/or secondary cell wall deposition between the genetically modified cell and an unmodified or wild type form of the same cell.

The rate and/or extent of cell wall deposition in the genetically modified cell may be modulated according to the method of the first aspect of the invention. Thus, the present invention also provides a genetically modified plant cell wherein the rate and/or extent of cell wall deposition in the genetically modified cell has been modulated according to the method of the first aspect of the invention.

The cell may comprise a modulated rate and/or extent of primary or secondary cell wall deposition. However, in one particular embodiment, the cell comprises a modulated rate and/or extent of secondary cell wall deposition.

Furthermore, the cell may comprise increased or decreased expression of a homeodomain/leucine zipper (HD-Zip) polypeptide relative to an unmodified form of the cell, leading to a decreased or increased rate and/or extent of cell wall deposition, respectively.

In one particular embodiment, the cell comprises an increased expression of the HD-Zip polypeptide and a decreased rate and/or extent of secondary cell wall deposition relative to an unmodified form of the cell.

In various embodiments, the HD-Zip polypeptide having modulated expression in the genetically modified cell is as hereinbefore described with reference to the first aspect of the invention.

In a further embodiment, the expression of the HD-Zip polypeptide is modulated by modulating the expression of a HD-Zip polypeptide-encoding nucleic acid in the plant cell.

Suitable HD-Zip polypeptide encoding nucleic acids, and methods for modulating their expression in a plant cell, include those hereinbefore described with reference to the first aspect of the invention.

As set out with respect to the first aspect of the invention, the modulated rate and/or extent of cell wall deposition in the cell may include the actual rate and/or extent of cell wall deposition by a plant cell and/or any process in the plant cell which is involved in or associated with cell wall deposition. Thus, any one or more of the following may be modulated in the cell: actual cell wall deposition, the expression of one or more cell wall biosynthetic enzymes (including secondary cell wall biosynthetic enzymes), the amount of one or more primary or secondary cell wall components in the plant cell wall, and the like.

Thus, in one embodiment, the modulated rate and/or extent of cell wall deposition in the plant cell comprises modulation of the expression of one or more secondary cell wall biosynthetic enzymes in the plant cell, as described above with reference to the first aspect of the invention.

In a further embodiment, the modulated rate and/or extent of cell wall deposition in the plant cell may comprise modulation of the rate and/or extent of lignin deposition in the cell wall of the plant cell, as described above with reference to the first aspect of the invention.

In a further embodiment, the size of the plant cell, as measured in at least one dimension, may also be modulated. In one particular embodiment the expression of the HD-Zip polypeptide is increased in the plant cell and this effects an increase in the size of the plant cell, as measured in at least one dimension.

The plant cell of the second aspect of the invention may be any cell from an organism of the kingdom Plantae. As such, the cell may be a bryophyte cell or a vascular plant cell. Generally, the cells used in accordance with the present invention include walled members of this kingdom. However, naturally non-walled members of the kingdom may be used and the present invention may be used to promote primary or secondary cell wall deposition.

In some embodiments, the cell is a monocotyledonous or dicotyledonous angiosperm plant cell or a gymnosperm plant cell. In one particular embodiment, the monocotyledonous plant cell, and in a further embodiment a cereal crop plant cell, as previously defined.

In a third aspect, the present invention provides a plant or a part, organ or tissue thereof comprising one or more cells according to the second aspect of the invention.

The plant of the third aspect of the invention may be any multicellular organism of the kingdom Plantae, including bryophytes and vascular plants. In some embodiments, the plant is a monocotyledonous or dicotyledonous angiosperm plant or a gymnosperm plant. In one particular embodiment, the plant is a monocotyledonous plant, and in a further embodiment a cereal crop plant, as previously defined.

As referred to herein, “a plant or a part, organ or tissue thereof” should be understood to include a whole plant or any part thereof. As such, this term may encompass whole plants, plant reproductive material or germplasm including seeds, vegetative plant tissue, harvested plant tissue, silage, cuttings, grafts, explants and the like.

The plants of the third aspect of the invention may exhibit an altered phenotype relative to a wild type form of the plant. For example, as detailed in Examples 2 and 3, plants which overexpress an HD-Zip II and thus have downregulated secondary cell wall deposition may exhibit a phenotype selected from dwarfism, altered leaf morphology, altered leaf colour, delayed flowering time, altered spike morphology, altered grain yield, altered grain morphology or altered fertility.

In a fourth aspect, the present invention provides a plant cell culture or plant tissue culture comprising one or more cells according to the second aspect of the invention.

A “plant cell culture” or “plant tissue culture” refers to cultured cells or tissues in any form, including, colonies, calli, embryogenic call, cultured embryos, cultured plantlets, suspension cultures and the like.

In a fifth aspect, the present invention provides a method determining and/or predicting the rate and/or extent of cell wall deposition in a plant, or a part, organ, tissue or cell thereof, the method comprising determining the expression of a homeodomain/leucine zipper (HD-Zip) polypeptide in the plant or a part, organ, tissue or cell thereof.

The method of the fifth aspect of the present invention may be used, among other things, to select a plant, or part, organ, tissue or cell thereof, which has a desired level of cell wall deposition. These plants may then be selected for breeding or other techniques (such as clonal propagation) to generate progeny plants having a desired level of cell wall deposition in one or more cells.

In addition, a plant or a part, organ, tissue or cell thereof may be selected for further downstream processing or application on the basis of the determined or predicted rate and/or extent of cell wall deposition.

The method of the fifth aspect may be used to determine and/or predict the rate and/or extent of cell wall deposition in a plant, or a part, organ, tissue or cell thereof. However, in one particular embodiment, the method is for determining the rate and/or extent of secondary cell wall deposition in a plant, or a part, organ, tissue or cell thereof.

In further embodiments, the HD-Zip polypeptide is a class II HD-Zip polypeptide, as hereinbefore defined with regard to the first aspect of the invention.

The method contemplates any means by which the expression of a HD-Zip polypeptide in a cell may be determined. This includes, for example, methods such as determination of the level and/or activity of an HD-Zip polypeptide in a cell and/or determining the expression of an HD-Zip polypeptide encoding nucleic acid in the plant, or a part, organ, tissue or cell thereof.

In one particular embodiment, the expression of the HD-Zip polypeptide is determined by determining the expression of a HD-Zip polypeptide-encoding nucleic acid in the plant or a part, organ, tissue or cell thereof. Suitable HD-Zip polypeptide encoding nucleic acids include those hereinbefore described with reference to the first aspect of the invention.

Methods for determining the level and/or pattern of expression of a nucleic acid or polypeptide are known in the art. Exemplary methods of the detection of RNA expression include methods such as quantitative or semi-quantitative reverse-transcriptase PCR (eg. see Burton et al., Plant Physiology 134: 224-236, 2004), in-situ hybridization (eg. see Linnestad et al., Plant Physiology 118: 1169-1180, 1998); northern blotting (eg. see Mizuno et al., Plant Physiology 132: 1989-1997, 2003); and the like. Exemplary methods for determining the expression of a polypeptide include Western blotting (eg. see Fido et al., Methods Mol Biol. 49: 423-37, 1995); ELISA (eg. see Gendloff et al., Plant Molecular Biology 14: 575-583); immunomicroscopy (eg. see Asghar et al., Protoplasma 177: 87-94, 1994) and the like.

This aspect of the invention may be utilised to select a plant or a part, organ, tissue or cell thereof on the basis of a determined and/or predicted relatively low or relatively high cell wall deposition. For example, relatively high expression of a homeodomain/leucine zipper (HD-Zip) in a plant or a part, organ, tissue or cell thereof, is associated with a relatively low rate and/or extent of cell wall deposition. Meanwhile, relatively low expression of a homeodomain/leucine zipper (HD-Zip) polypeptide in a plant or a part, organ, tissue or cell thereof, is associated with a relatively high rate and/or extent of cell wall deposition.

In one particular embodiment, increased expression of the HD-Zip polypeptide in a plant or a part, organ, tissue or cell thereof is associated with a decrease in the rate and/or extent of secondary cell wall deposition in a plant cell.

In the method of the fifth aspect of the invention, the rate and/or extent of cell wall deposition determined and/or predicted in accordance with the method may include the actual rate and/or extent of cell wall deposition by a plant cell and/or the rate and/or extent of any process in the plant cell which is involved in or associated with cell wall deposition. Thus, by determining the expression of a homeodomain/leucine zipper (HD-Zip) polypeptide in the plant or a part, organ, tissue or cell thereof, any one or more of the following may be determined and/or predicted in the cell: actual cell wall deposition, the expression of cell wall biosynthetic enzymes, the amount of one or more primary or secondary cell wall components in the plant cell wall, and the like.

Thus, in one embodiment, the present invention provides a method for determining and/or predicting the expression of one or more secondary cell wall biosynthetic enzymes in a plant or a part, organ, tissue or cell thereof, the method comprising determining the expression of a homeodomain/leucine zipper (HD-Zip) polypeptide in the plant or a part, organ, tissue or cell thereof.

In one particular embodiment an increase in the expression of the HD-Zip polypeptide in the plant or a part, organ, tissue or cell thereof is associated with a decrease in the expression of one or more secondary cell wall biosynthetic enzymes in the plant or a part, organ, tissue or cell thereof.

In a further embodiment, the present invention provides a method for determining and/or predicting the rate and/or extent of lignin deposition in a cell wall of a plant or a part, organ, tissue or cell thereof, the method comprising determining the expression of a homeodomain/leucine zipper (HD-Zip) polypeptide in the plant or a part, organ, tissue or cell thereof.

In one particular embodiment, an increase in the expression of the HD-Zip polypeptide in the plant or a part, organ, tissue or cell thereof is associated with a decrease in the rate and/or extent of lignin deposition in a cell wall of the plant or a part, organ, tissue or cell thereof.

In further embodiments, determining the expression of a homeodomain/leucine zipper (HD-Zip) polypeptide in the plant or a part, organ, tissue or cell thereof further determines or predicts the size of one or more cells, as measured in at least one dimension, in the plant or a part, organ, tissue or cell thereof. In a particular embodiment, an increase in the expression of the HD-Zip polypeptide in the plant or a part, organ, tissue or cell thereof is associated with an increase in the size of a plant cell in the plant or a part, organ, tissue or cell thereof, as measured in at least one dimension.

The method of the fifth aspect of the invention may be practiced on any plant cell, as hereinbefore defined. However, in some embodiments, the cell may be a monocotyledonous or dicotyledonous angiosperm plant cell or a gymnosperm plant cell. In one particular embodiment, the monocotyledonous plant cell, and in a further embodiment a cereal crop plant cell.

Although cereal crop plants are particularly suitable monocotyledonous plants, the other monocotyledonous plants may also be used, such as other non-cereal plants of the Poales, specifically including pasture grasses such as Lolium spp.

Finally, reference is made to standard textbooks of molecular biology that contain methods for carrying out basic techniques encompassed by the present invention, including DNA restriction and ligation for the generation of the various genetic constructs described herein. See, for example, Maniatis et al., Molecular Cloning: A Laboratory Manual (Cold Spring Harbor Laboratory Press, New York, 1982) and Sambrook et al. (2000, supra).

The present invention is further described by the following non-limiting examples:

### BRIEF DESCRIPTION OF THE FIGURES

FIG. 1 shows the phenotype of transgenic barley plants with constitutive overexpression of TaHDZipII-1. A and B—T₀ plants; C, D and E—T₁ plants; F and G—T₂ plants. Numbers reflect number of lines and sub lines. Wild type plants and transgenic plants transformed with empty vector were used as a control.

FIG. 2 shows transgene expression in T₀ transgenic lines (A) and T₁ progeny of line 3 (weak phenotype) and line 5 (strong phenotype) (B) using northern blot hybridization.

FIG. 3 shows the number of shoots in control and transgenic plants at the beginning of flowering. C1-C3—control plants transformed with empty vector. Numbers correspond to numbers of T₁ progeny of line 5.

FIG. 4 shows leaf size, color and shape in control and transgenic plants. C1-C3—control plants transformed with empty vector. Numbers correspond to numbers of T₁ progeny of line 5.

FIG. 5 shows the delay in flowering of transgenic plants in comparison with control plants. C1-C3—control plants transformed with empty vector. Numbers correspond to numbers of T₁ progeny of line 5. Delay is shown in days; the flowering of the first spike of C1 plant (observed release of pollen) is considered as a 0 day of flowering.

FIG. 6 shows spike development in control and transgenic plants. A—developing spikes at similar stages after anthesis; B—mature spikes; C—grains from control plant and transgenic T₁ plants.

FIG. 7 shows grain development in control and transgenic plants. A-F—development of unpollinated gametophyte of transgenic (T) and normally fertilized gametophyte of control (C) plant at A—2, B—4, C—12, E—15, and F—20 days after pollination. G—dark not opened anthers of transgenic (T) and normally developed anthers of control (C) plants at the same stage of development. H—grain from transgenic (T) and control (C) plant at 34 DAP. Lemma and palea of transgenic plant is undeveloped.

FIG. 8 shows the size and shape of epidermal cells of control and transgenic plants analyzed using scanning electron microscopy. A—epidermis of stem; B—epidermis of leaf. White arrows show upper and, if it is possible, lower borders of cells. Epidermal cells of stem in transgenic plants are 2.5-3 fold longer than in control plants and both borders cannot be shown on the same picture at this magnification.

FIG. 9 shows the morphology of stem and leaf of control and transgenic plants. A—morphology of stem; vascular tissues with increased number of xylem vessels in transgenic plants are in frames; xylem vessels are shown with arrows. B—morphology of leaf; bundle sheath extension cells with collapsed cell walls in transgenic plants are in frames.

FIG. 10 shows the visualization of lignin by staining of different tissues of transgenic and wild type plants with phloroglucinol. A—vascular bundles of stem of control and transgenic plant; B—vascular tissues of leaf; C—grain cross-sections and isolated lemma of transgenic and control plants. Red arrows show deposition of lignin in different tissues of wild type plant and lover amount or absence of lignin in the same tissues of transgenic lines. In panel C, T₂ progeny of two independent transgenic lines were used to demonstrate correlation of lignin levels with the strength of plant phenotype: 3-3-4—line with weak phenotype and 5-12-2—line with strong phenotype. VT—vascular tissue; S—sclerenchyma; L—lemma.

FIG. 11 shows the expression of potential downstream genes in wild type (WT) and T₁ progeny of transgenic lines (3-3, 5-2, 5-9) demonstrated by quantitative PCR. A—genes involved in biosynthesis of secondary cell walls; B—some other genes related to cell wall biosynthesis. Levels of expression are shown in relative units (copy number per μl of RNA solution).

FIG. 12 shows the chlorophyll content in control (G6, WT) and transgenic (G49) plants using SPAD-meter measurement. Panel A shows the T1 progeny of the G49-5 line; while panel B shows the T2 progeny of the G49-5 and G49-3 lines. Darker bars represent control plants, while the lighter bars represent transgenic plants.

FIG. 13 shows scanning electron micrographs of leaves from wild type (WT) and transgenic (Line 49-5) plants.

FIG. 14 shows autofluorescence of transverse leaf (A, B) and anther (C-F) sections, showing a decreased level of lignin in the cell walls of vascular tissue (indicated with arrows) in transgenic barley plants having up-regulated expression of TaHDZipII-1.

FIG. 15 shows Immunohistochemical analysis of wild type wheat for the identification of TaHDZipII-1 in shoot (A, B) and leaf (C-F) tissues of non-transgenic wheat. Serial transverse sections of shoot and leaf were stained using a mix of several TaHDZipII-1 specific monoclonal antibodies (A, C and E) and the pre-immune serum (B, D and F). The images shown are illuminated under UV light. The localisation of TaHDZipII-1 in the vascular tissue of shoots and the phloem of leaves is indicated with arrows.

### EXAMPLE 1

### General Materials and Methods

### (i) Plasmid Construction

The full-length coding region (CDS) of the TaHDZipII-1 cDNA (Acc No DQ353857—Lopato et al., Plant Methods 2: 3, 2006) was cloned into the donor vector pENTR-D-TOPO (Invitrogen). The cloned insert was sequenced and re-cloned by recombination into the pMDCUbi (pUbi) vector. pUbi is a derivative of pMDC32 vector (Curtis and Grossniklaus, Plant Physiol. 133: 462-469, 2003) in which 2×35S promoter was cut out using HindIII and KpnI restriction sites and replaced with maize polyubiquitin promoter (Christensen et al., Plant Mol Biol. 18(4): 675-89, 1992).

The resulting construct was designated pUbi-TaHDZipII-1 and was transformed into the Agrobacterium tumefaciens strain AGL1 by electroporation. The presence of the plasmid in selected bacterial clones was confirmed by PCR using specific primers (Table 2) derived from the CDS of the plant gene.

### (ii) Plant Transformation and Growth Conditions

Bread wheat (Triticum aestivum L. cv. Chinese Spring) and barley (Hordeum vulgare L. cv. Golden Promise) plants were grown in the glasshouse with day temperatures of 18-25° C. and night temperatures of 18-21° C., with a 10-13 h photoperiod. pUbi-TaHDZipII-1 was transformed into barley (Hordeum vulgare L. cv. Golden Promise) using Agrobacterium tumefaciens-mediated transformation developed by (Tingay et al., Plant Journal 11: 1369-1376, 1997) and modified by (Matthews et al., Molecular Breeding 7: 195-202, 2001). Transgenic plants were grown in a PC2 glasshouse with 10-hr light photoperiod. Plant phenotype was studied in T₀, T₁ and T₂ generations of several transgenic lines.

(iii) mRNA Isolation and Hybridization Techniques

Transgene integration in barley plants was confirmed by Southern blot hybridization. Genomic DNA from selected barley lines was digested with Xho1 and probed with the coding sequence of hygromycin phosphotransferase.

Total RNA was isolated from wheat and barley samples using TRI REAGENT (Molecular Research Centre, Inc., Cincinnati, Ohio) and used in Northern blot hybridization. Pools of single strand cDNAs for Q-PCR were prepared using SuperScript III reverse transcriptase (Invitrogen).

### (iv) Quantitative PCR Analysis

The forward primer for TaHDZipII-1 was designed for the wheat variety Chinese Spring using the 3′UTR sequence of the cDNA. To provide specific recognition of transgene cDNA the reverse primer sequence was selected from Nos terminator of vector plasmid (Table 2).

The primer pairs for cell wall enzymes were designed for the barley variety Golden Promise using 3′UTR sequences (Table 2).

TABLE 2
Q-PCR primers

| Target | Forward primer | Reverse primer |
|---|---|---|
| TaHDZipII-1 | GTACAGAGACACCGGAGCAAC | TTGCCAAATGTTTGAACGATC |
| | (SEQ ID NO: 3) | (SEQ ID NO: 4) |
| Laccase 1 | TCATTGCCAGAGTGTTGTCAG | CTAGGCTTTATTTAGCGATAC |
| | (SEQ ID NO: 5) | (SEQ ID NO: 6) |
| Laccase 2 | TTCCTCCCCCTCCCGAAGATC | AAGAACGTATTTCCGCTATTC |
| | (SEQ ID NO: 7) | (SEQ ID NO: 8) |
| HvCesA4 | GCCCAAGGGACCCATTCTTA | TTAGAACTTGGAACCCCCCA |
| | (SEQ ID NO: 9) | (SEQ ID NO: 10) |
| HvCesA7 | TGAGCAGCTGCCGTTGCTTGG | AATAGTAGCCTACATCACCTCTG |
| | (SEQ ID NO: 11) | (SEQ ID NO: 12) |
| HvCesA8 | ACAGTTTGGACGCAAGTTTTGTATT | CGGTCCTCTGTTCAATTCTTGTTTA |
| | (SEQ ID NO: 13) | (SEQ ID NO: 14) |
| HvCesA1 | TGTGGCATCAACTGCTAGGAAA | CGTACAAAGTGCCTCATAGGAAA |
| | (SEQ ID NO: 15) | (SEQ ID NO: 16) |
| HvCesA3 | ACACGAGTCACTGGGCCAGA | CTGGTAAACTAGTCACCCGCTGA |
| | (SEQ ID NO: 17) | (SEQ ID NO: 18) |

The Q-PCR amplification was performed in an RG 2000 Rotor-Gene Real Time Thermal Cycler (Corbett Research, NSW, Australia) using QuantiTect SYBR Green PCR reagent (Qiagen, VIC, Australia), as described in Burton et al. Plant Physiol. 134(1):224-36, 2003).

The Rotor-Gene V4.6 software (Corbett Research) was used to determine the optimal cycle threshold (CT) from dilution series and the mean expression level and standard deviations for each set of four replicates for each cDNA were calculated.

### (v) Microscopy

Stems and leaves of control and transgenic plants were fixed in 0.25% glutaraldehyde, 4% paraformaldehyde, 4% sucrose and 1 M sodium phosphate. After fixation, plant material was rinsed (2-3 changes in 8 hours to remove fixative) in 1 M sodium phosphate. Tissues were rinsed and dehydrated in a successive ethanol series (70, 90, 95, 100%), and infiltrated step-wise with xylene (25, 50, 75, 100% in ethanol). 7 μm thick sections were stained with 0.01% (w/v) toluidine blue in 0.1% aqueous sodium tetraborate for 1-5 mins.

To visualise lignin, hand-cut sections from fresh barley stems or leaves were stained with phloroglucinol-HC1. Each section was covered with 2-3 drops of 1% w/v solution of phloroglucin in 95% ethanol, followed by the addition of 1 drop of 25% (w/v) HC1. The results were observed using a Laser Dissection microscope (Leica AS LMD).

### (vi) Scanning Electron Microscopy (SEM)

Samples for scanning electron microscopy were examined in a Philips XL30 field-emission gun scanning electron microscope (SEM) fitted with an Oxford CT1500 cryo-transfer system.

Samples were prepared by slicing millimetre-sized sections from an appropriate part of a plant (e.g. leaf, stem, etc.). Every effort was made to keep the plant hydrated before and during preparation for SEM examination. The sections of plant were mounted on SEM holders either by using a clamping vice attached to the holder or with OTR. The samples were rapidly frozen by plunging into a slush of liquid and solid nitrogen and then transferred under vacuum while frozen to the cryo-transfer chamber. Whilst maintained at a temperature of approximately −130° C. some samples where fractured to expose internal structure. After fracturing, a small amount of water (a layer approximately 1 μm thick) was removed from the samples by heating to −92° C. for 120 secs. A similar water removal process was applied to samples that were not fractured so as to remove any atmospheric water that may have been frozen on the surface of the sample during processing. After water removal, the samples were cooled to less than −110° C. and coated with approximately 15 nm of Pt to make them electrically conductive.

The coated samples were transferred to the SEM chamber where they were maintained at a temperature of approximately −150° C. throughout the SEM imaging process.

### EXAMPLE 2

### Phenotypic Effects of Constitutive Overexpression of TaHDZipII-1

H. vulgare cv Golden Promise was used for transformation because of the relatively high efficiency of transformation using Agrobacterium-mediated transformation.

A modification of pMDC32 vector, designated pMDCUbi or pUbi was used for transformation. In this vector, the 2×35S promoter was exchanged for polyubiquitin promoter, which has been shown to be one of the strongest constitutive promoters isolated from monocotyledonous plants and more efficient than the cauliflower mosaic virus 35S promoter for transgene overexpression in grasses.

Cloning of the coding region of TaHDZipII-1 into the pUbi vector under polyubiquitin promoter generated the pUbi-TaHDZipII-1 construct, which was used for plant transformation. Plant transformation was repeated twice and resulted in 17 independent transgenic lines selected on hygromicin.

Transgene integration was confirmed in 10 lines by Southern blot hybridization using the coding region of hygromicin phosphotransferase as a probe. It was found that transgenic lines generally contained from 1 to 6 copies of the transgene.

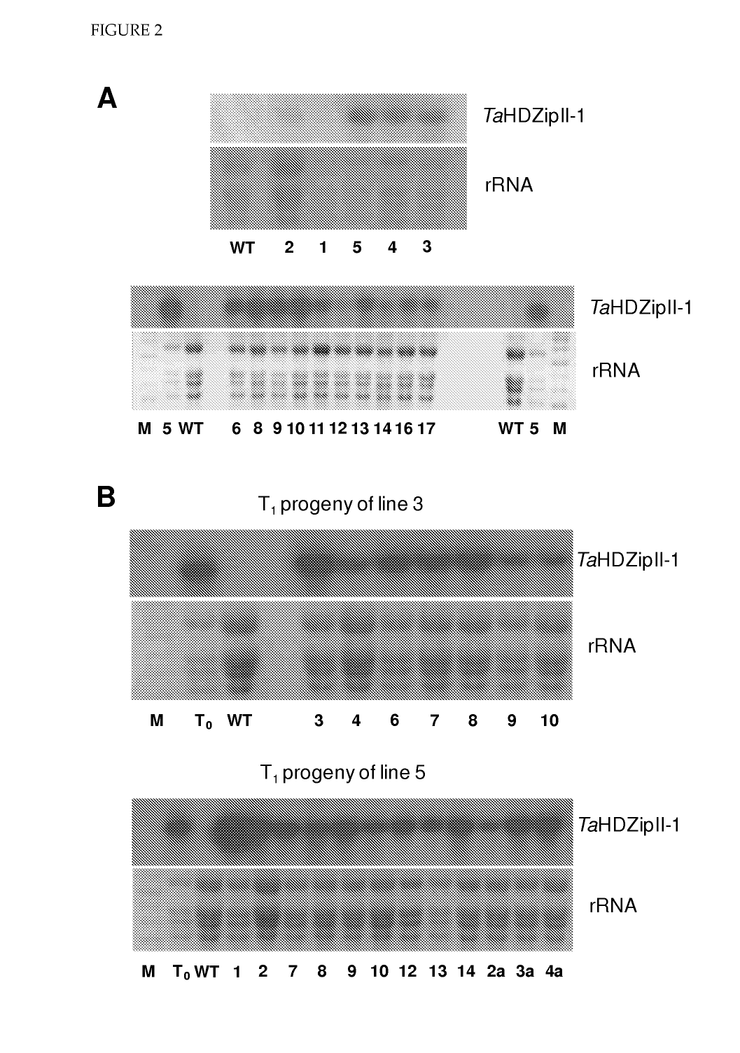

Transgene expression was observed in 16 of 17 T₀ lines (See Table 3 and FIG. 2). The T₀ lines could be divided into three groups: 4 plants with very strong transgene expression and a very strong phenotype; 3 plants with relatively strong transgene expression and a mild phenotype; and 6 plants with weak transgene expression and no or a very weak phenotype, which was observed mainly during flowering (length and shape of spike).

TABLE 3
Summary of transgene
expression in T0 barley lines

| | | Transgene | |
|---|---|---|---|
| T0 | Transgene | expression | Strength |
| identifier | copy number | strength | of phenotype |
| 1 | 4 | − | − |
| 2 | no data | + | − |
| 3 | 1-2 | ++ | + |
| 4 | 3-5 | ++ | died before |
| | | | flowering |
| 5 | 4-5 | +++ | +++ |
| 6 | 4-5 | ++ | ++ |
| 8 | 5-6 | +++ | +++ |
| 9 | 5 | +++ | +++ |
| 10 | no data | +++ | ++ |
| 11 | 3 | + | + |
| 12 | no data | + | + |
| 13 | 4 | ++ | ++ |
| 14 | 2 | + | + |
| 16 | no data | + | + |
| 17 | no data | + | + |

No clear correlation was found between copy number and strength of phenotype. However, all T₀ lines showed generally the same phenotype, except line 4, which had extremely slow growth and died before flowering.

Characteristic features of the observed phenotype were slow growth, delayed flowering and slow seed development. As a result, the life cycle of the transgenic barley exceeded one year.

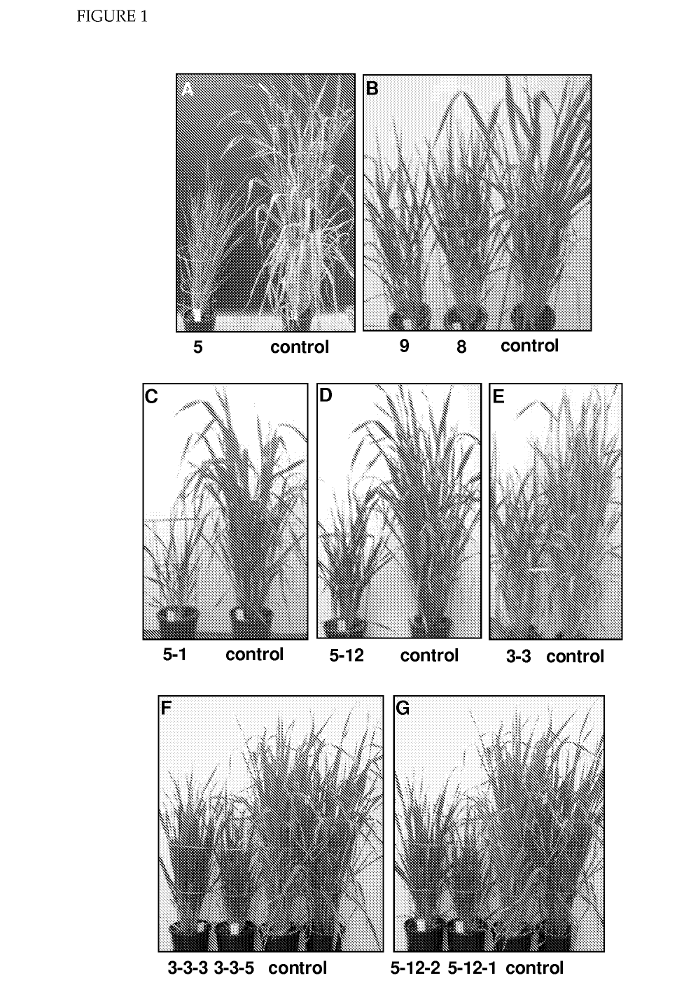

The phenotype observed in T₀ lines consistently reappeared in two consecutive generations of transgenic plants (see FIG. 1).

All analysis has been performed on T₁ and T₂ lines with confirmed transgene expression and a clear phenotype.

Transgenic plants with a strong phenotype demonstrated dwarfism. They grew substantially slower than control plants and their size at flowering time did not exceed two thirds of the size of control plants (FIG. 1). Also the size of the transgenic plants was found to generally inversely correlate with the strength of transgene expression in the plant.

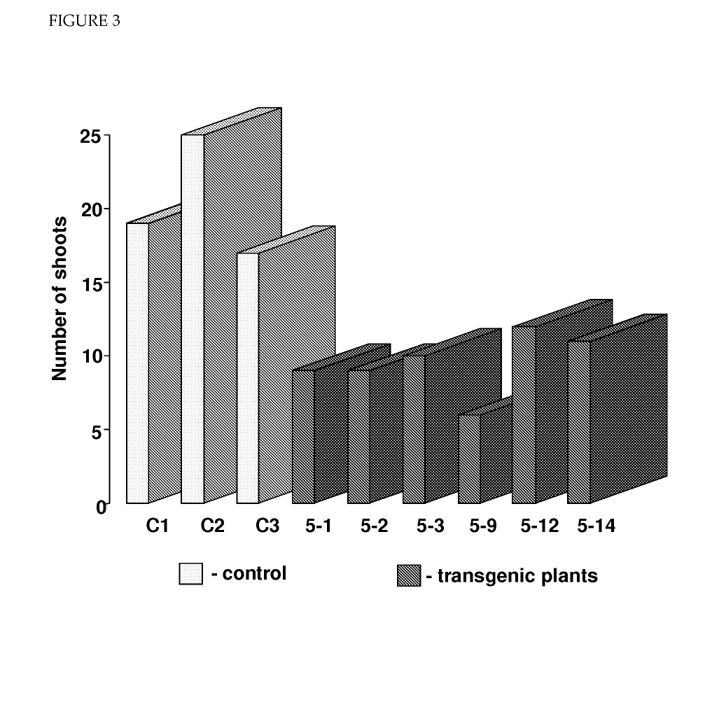

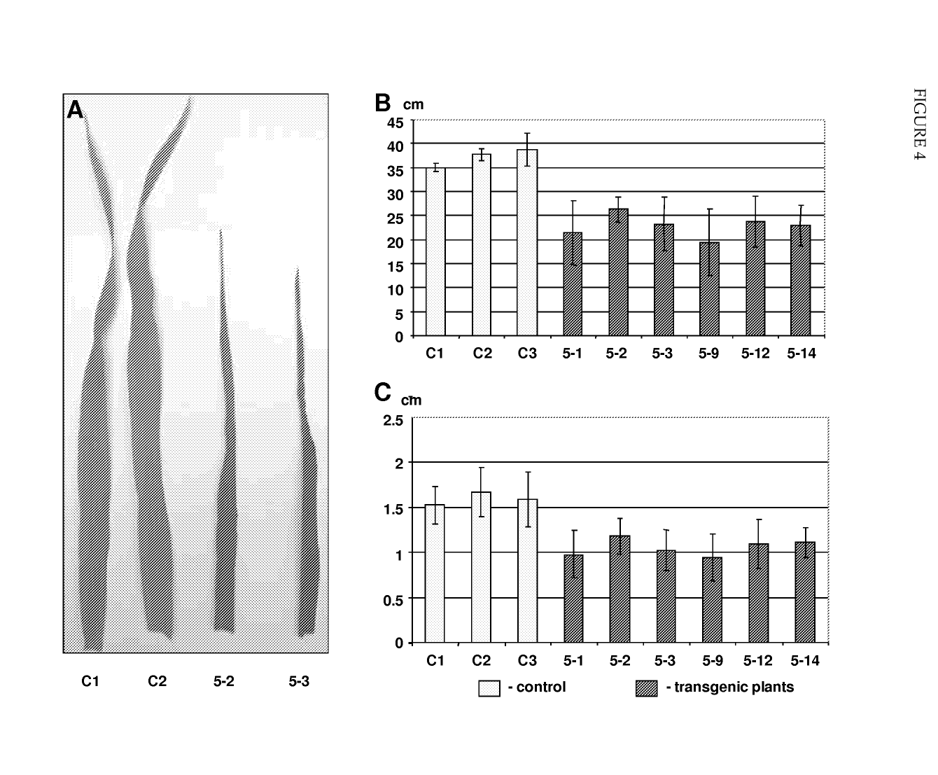

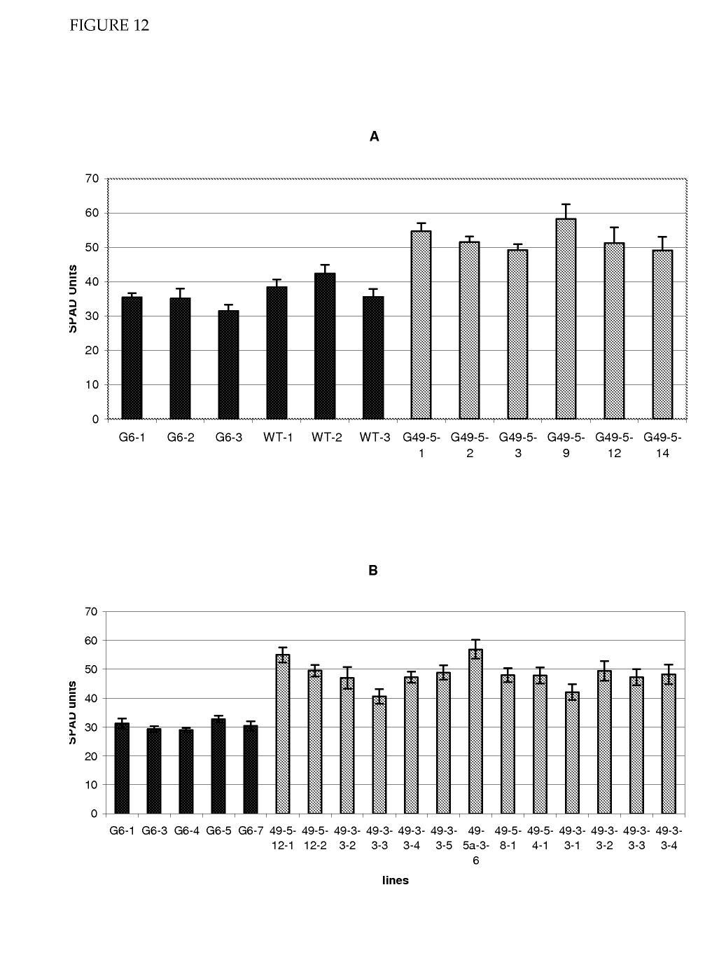

Transgenic plants produced about 50% fewer shoots (FIG. 3). Also, the leaves of transgenic plants were smaller and thinner than leaves of control plants, always erect and dark green in color (FIG. 4). Analysis of chlorophyll content in leaves using a spadmeter revealed a 12-46% higher chlorophyll concentration in transgenic plants than in control plants (FIG. 12).

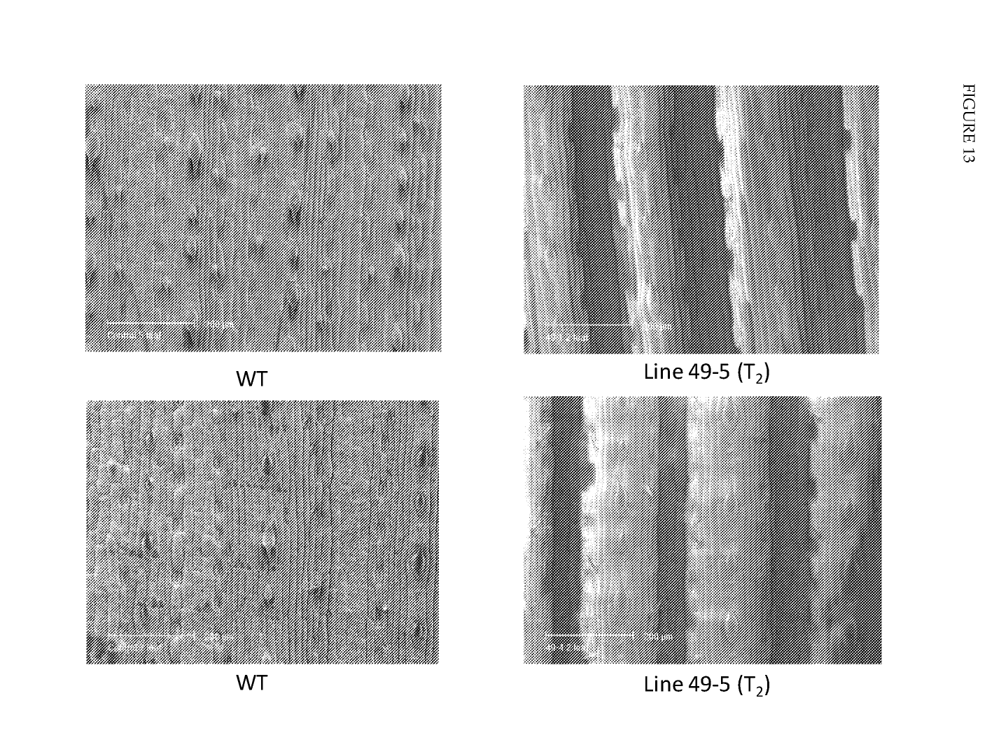

A possible explanation of the dark color and erect nature of the leaves in the transgenic plants comes from SEM analysis. It was found that transgenic plants have thicker ribs with smaller distance between them. This subsequently changes the architecture of leaf, particularly leaf thickness and light transparence and reflection, which can influence the results of spadmeter measurements (FIG. 13).

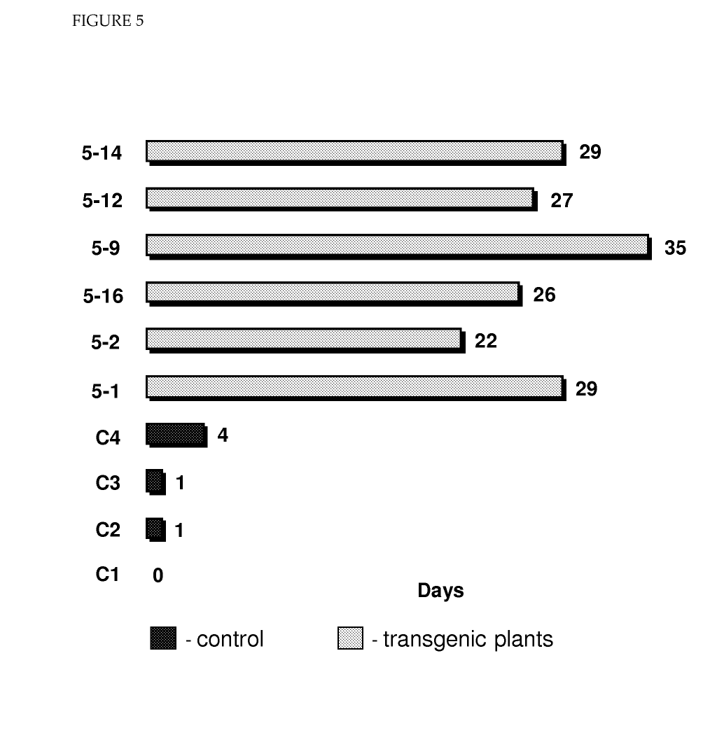

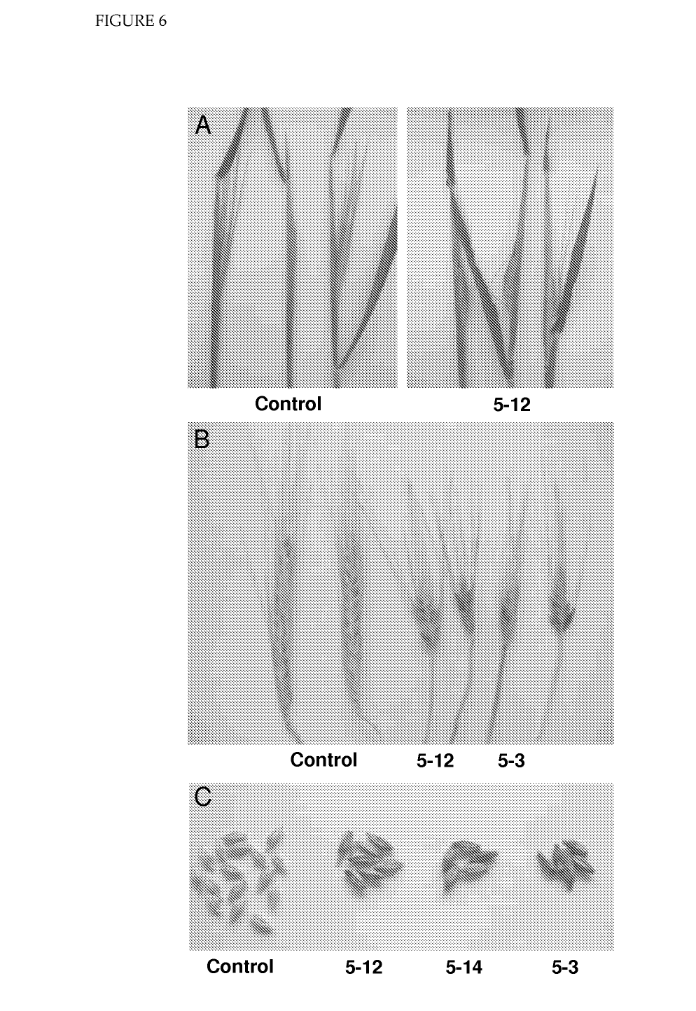

Transgenic plants also exhibited a 4-5 week delay in flowering (FIG. 5). Spikes of the transgenic plants were generally smaller than the spikes of control plants; and their shape and size strongly correlated with the level of TaHDZipII-1 overexpression in the plant. In those lines with the strongest phenotype, spikes were three-fold shorter than control spikes and had an oval-round shape. The stem under the spike was also shorter than the leaf sheath, and this may explain why one side of spikes was not released from sheath of flag leaf (FIG. 6A).

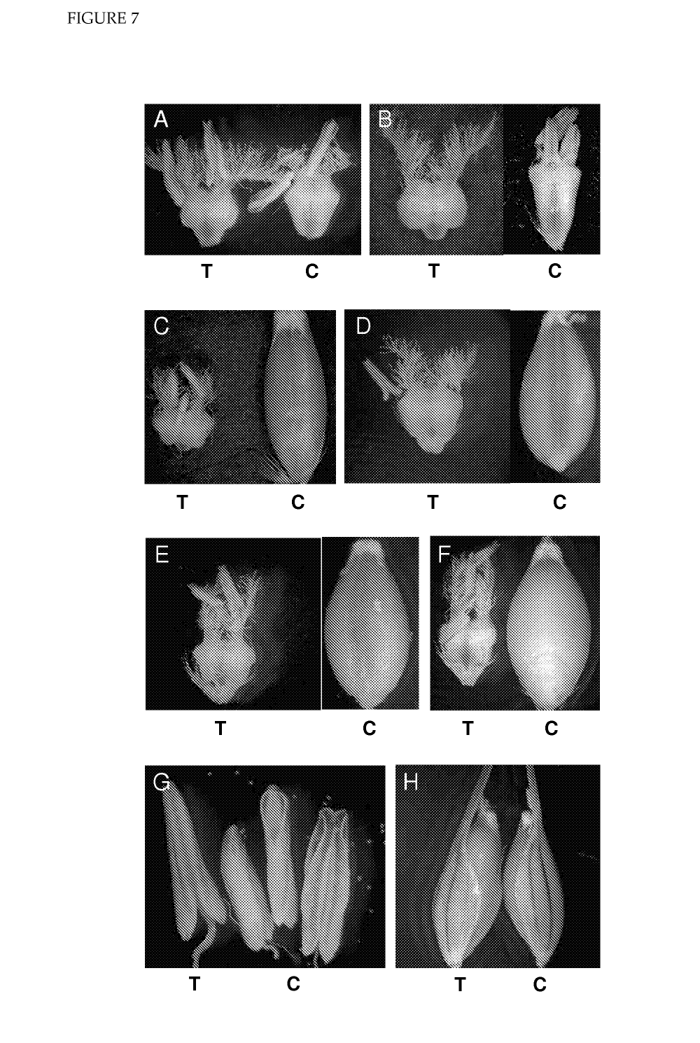

Few grains in the spikes of the transgenic plants developed to maturity. Development of most grains was either not started or was aborted during the first 4 Days After Pollination (DAP). One of the possible reasons for this may be male sterility as a result of abnormal anther development, which lead to their inability to open and release pollen (FIG. 7G).

The number of developing grains was lower in first flowering spikes, but higher in the small side spikes developed at the end of flowering. Successfully fertilized transgenic grain grew to the same size as control grain. In contrast to grain from control plants, a large part of the transgenic grain was exposed due to very short, underdeveloped lemma and palea in the transgenic plants (FIG. 7H).

Mature transgenic grain was also darker than grain of control plants (FIGS. 6B and 6C). About 30% of the transgenic grain was unable to germinate.

### EXAMPLE 3

### Cell Size and Morphology in Transgenic Plants

Since transgenic plants were smaller in size than control plants it was expected that they would also have smaller cells. Surprisingly, it was observed that at least some cell types are considerably longer in transgenic plants than in control plants.

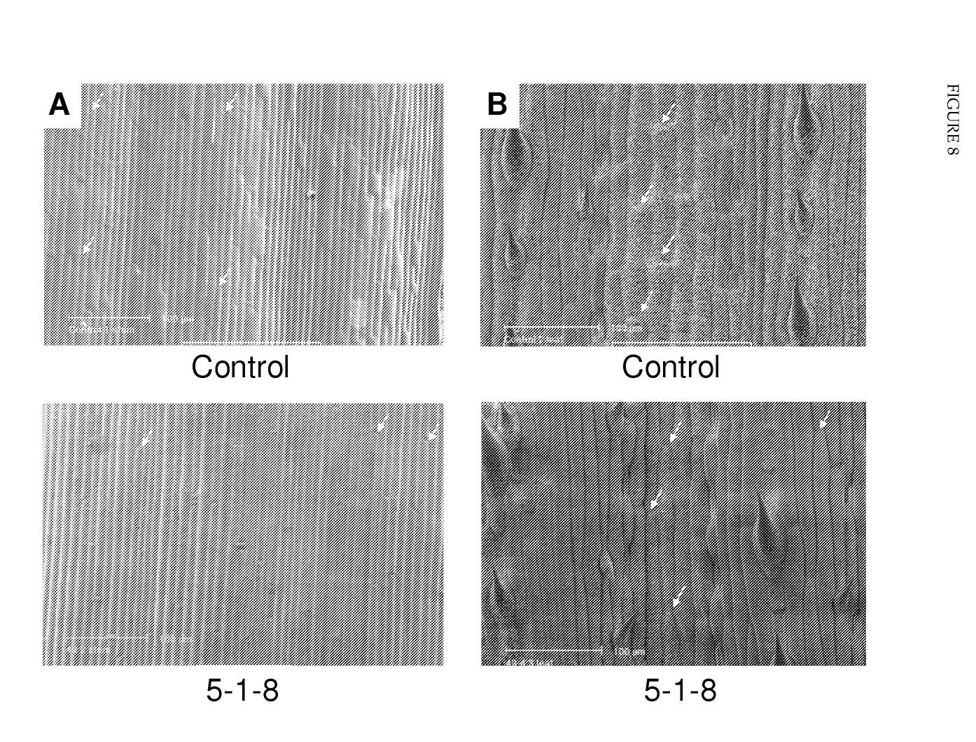

SEM analysis demonstrated that stem epidermis cells in transgenic plants are 2.5-3 fold longer than the same cells in control plants (FIG. 8A). Bulliform cells of the leaf epidermis were about 1.5 fold thinner and longer in transgenic plants (FIG. 8B).

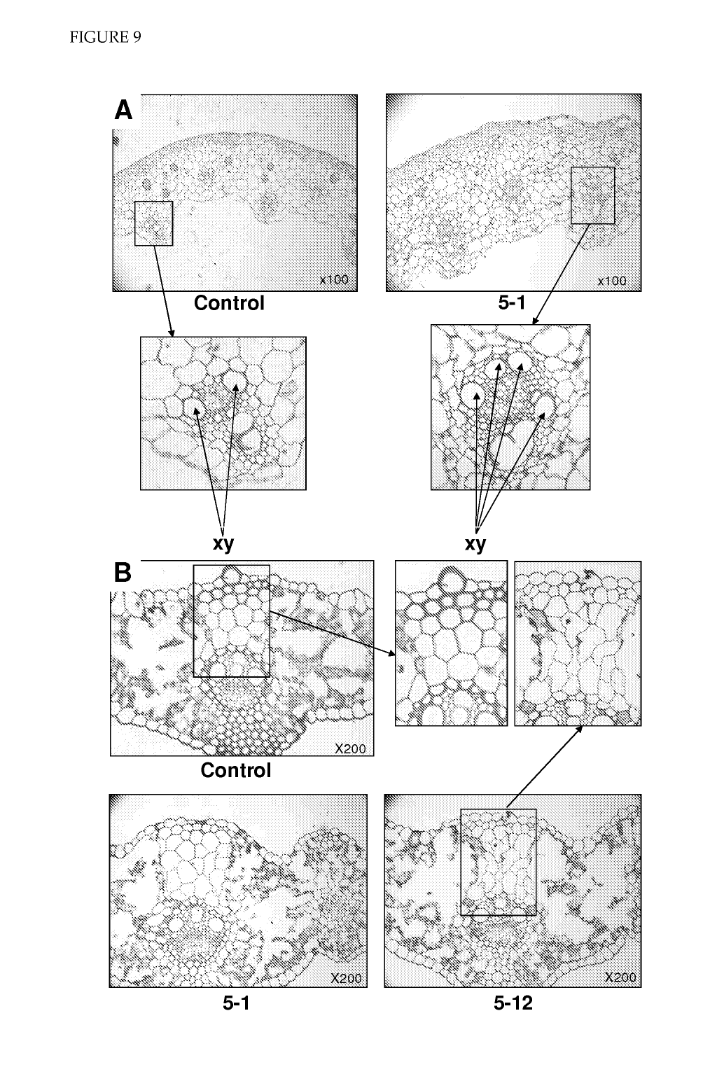

Morphological studies also demonstrated an increased number of xylem cells and some other types of cells in vascular system of the stem (FIG. 9A). Cells of the bundle sheath extension in leaves were also found to have an unusual cell wall shape. These cells in the transgenic plants seemed to have thinner cell walls, which partially collapsed under pressure of cells from adjacent tissues (FIG. 9B). The same group of cells in leaves of control plants always had normal near round shape. The strength of this phenotype correlated with the strength of transgene expression.

It was observed that the collapsed cells in the transgenic plants reported here had very similar shape to collapsed cells described earlier for irregular xylem (irx) mutants in Arabidopsis (Turner and Somerville, Plant Cell 9(5): 689-701, 1997). These Arabidopsis mutants have mutations in genes encoding several different enzymes responsible for biosynthesis of secondary cell walls and particularly lignin. However, all of them have very similar phenotypes. Interestingly, other features of irx mutant phenotype are dwarfism, dark green color and sterility (Brown et al., Plant Cell 17(8): 2281-95, 2005), which resemble the phenotype of the transgenic barley plants with overexpressed TaHDZipII-1.

### EXAMPLE 4

### Regulation of Lignin Biosynthesis in Plants with Overexpressed TaHDZipII-1

Based on the observation that overexpression of TaHDZipII-1 generated a phenotype similar to irx mutants in Arabidopsis (which have lower amount of lignin and cellulose than wild type plants) the transgenic plants that overexpressed TaHDZipII-1 were tested for the presence of lignin using diagnostic staining with phloroglucinol-HCl.

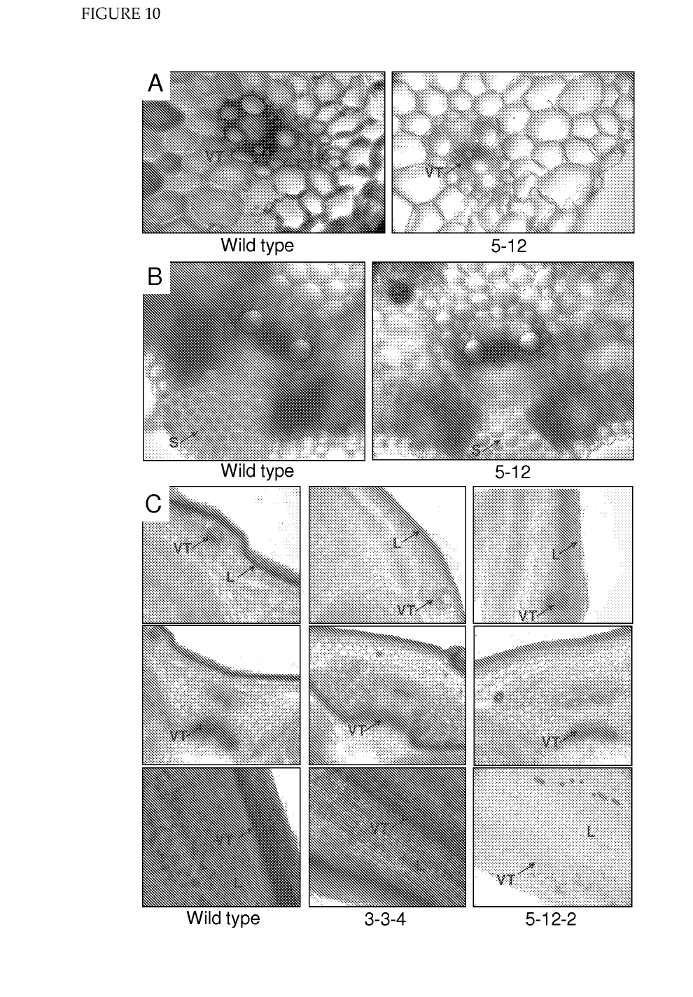

Phloroglucinol is indicative of ciniferaldehyde end-groups in guaiacyl residues (Clifford, J. Chromatogr. 94: 321-324, 1974). Staining of barley stem hand-cut sections localized lignin in the vascular tissue of control plants. However, no lignin was found in the vascular tissue of transgenic plants with a strong phenotype and a high level of TaHDZipII-1 overexpression (FIG. 10A).

Staining of leaf tissue also demonstrated an absence of lignin in cells of the vascular sheath extension in the transgenic plants (FIG. 10B).

A large amount of lignin was found in the lemma, palea and vascular tissue of developing and mature grain of control plants. However, the amount of lignin in lemma and vascular tissue of grain of transgenic plant inversely correlated with level of transgene expression and was very low plants with a strong phenotype (FIG. 10C).

### EXAMPLE 5

### Genes Regulated by TaHDZipII-1 in Transgenic Plants

As the phenotype of TaHDZipII-1 plants resembled the phenotype of irx mutants, Q-PCR was used to compare the expression levels of some genes involved in cell wall biosynthesis in control and transgenic plants.

The expression of genes encoding two types of enzymes, cellulose synthases and laccases, was tested using quantitative PCR (Q-PCR). The first group of enzymes comprised cellulose synthases class A (CesA), which are enzymes involved in cellulose biosynthesis.

Two groups of coordinately transcribed CesA genes were identified in barley (Burton et al., 2004, supra). One of them contains HvCesA4 (Acc. No. AY483154), HvCesA7 (SEQ ID NO: 19) and HvCesA8 (Acc. No. AY483156). It was contemplated that these may be involved in the biosynthesis of secondary cell walls.

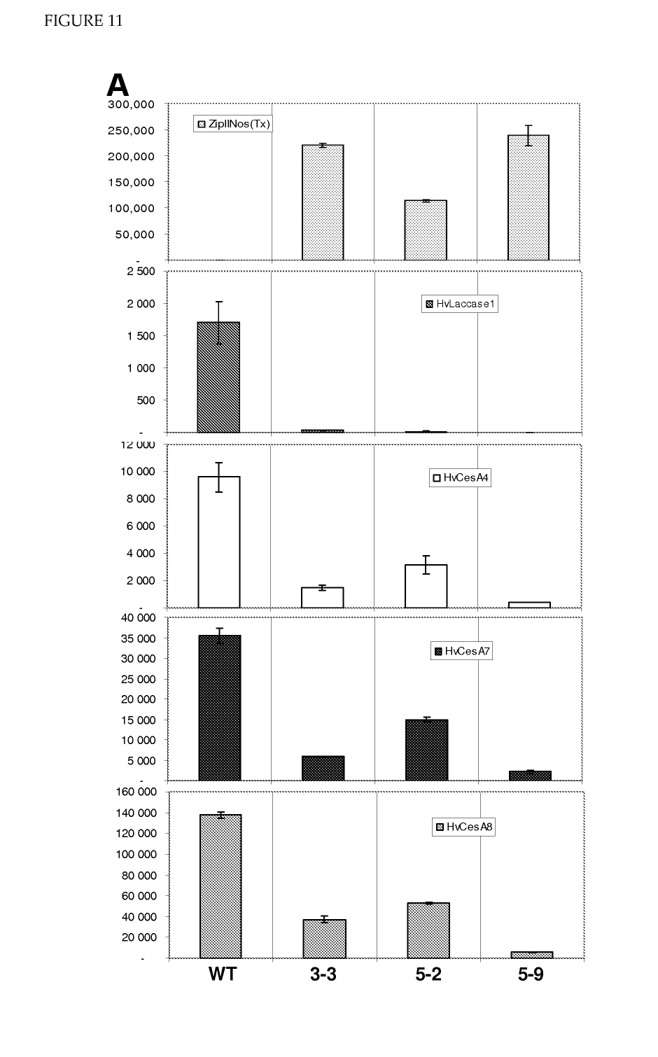

Expression of these three CesA genes was strongly down regulated in a coordinated way in all three tested transgenic plants (two independent lines) (FIG. 11). However, no substantial changes in transcript number was found in transgenic plants in comparison with control plants for two other CesA genes, namely HvCesA1 (Acc. No. AY483150) and HvCesA3 (Acc. No. AY483151) (data not shown).

One laccase (laccase 1, EST Acc. No. AL501631) was found to be coordinately expressed with the CesA genes from the first group in different tissues. Total repression of transcript synthesis of laccase 1 was detected in all tested transgenic lines (FIG. 11). The expression level of another laccase (laccase 2 EST Acc. No. CX628320) was slightly increased in transgenic plants, but remained below the border of sensitivity of Q-PCR.

### EXAMPLE 6

### Autofluorescence in TaHDZipII-1-Overexpressing Transgenic Plants

Autofluorescence may be used as an indication of lignin deposition in plant cell walls. Autofluoresence was examined in control (wild type) plants and in HD-Zip II overexpressing plants. Semi-thin sections (7 μm thick) were prepared on glass slides and imaged under Laser Dissection microscope (Leica AS LMD). A filter BP 355-425 nm was used as excitation filter and fluorescence was detected at >470 nm.

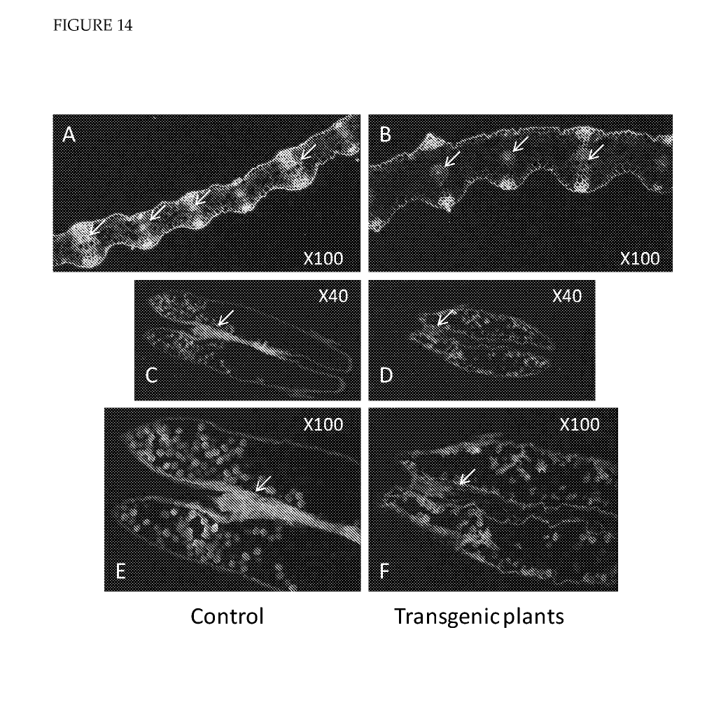

The results of the autofluorescence assay are shown in FIG. 14.

### EXAMPLE 7

### Immunolocalisation of TaHDZipII-1 in Wheat Tissues

For light microscopy (LM), semi-thin sections (7 μm thick) were prepared on poly-L-lysine coated slides (Poly-Prep™, Sigma-Aldrich; or SuperFrost® Plus or UltraPlus, Menzel GmbH supplied by Lomb Scientific; or ProbeOn Plus, FisherBiotech). Sections were dewaxed, rehydrated and pre-incubated in PBS buffer solution (pH 7.2) containing 1% (w/v) bovine serum albumin (BSA, Merck) for 40 min to block non-specific labelling. The sections were then incubated for 1 h with primary mouse anti-TaHDZipII-1 monoclonal antibodies solution. Dilutions of antibodies in PBS containing 1% (w/v) BSA were: 1:50, 1:100 and 1:200. The sections were then washed extensively with PBS-1% BSA and subsequently incubated for 2 h in the dark with goat anti-mouse fluorophore-conjugated secondary antibody. Sections were washed three times with PBS containing 1% (w/v) BSA. The BP 355-425 nm filter was used as an excitation filter and fluorescence was detected at >470 nm. All incubations were done at room temperature.

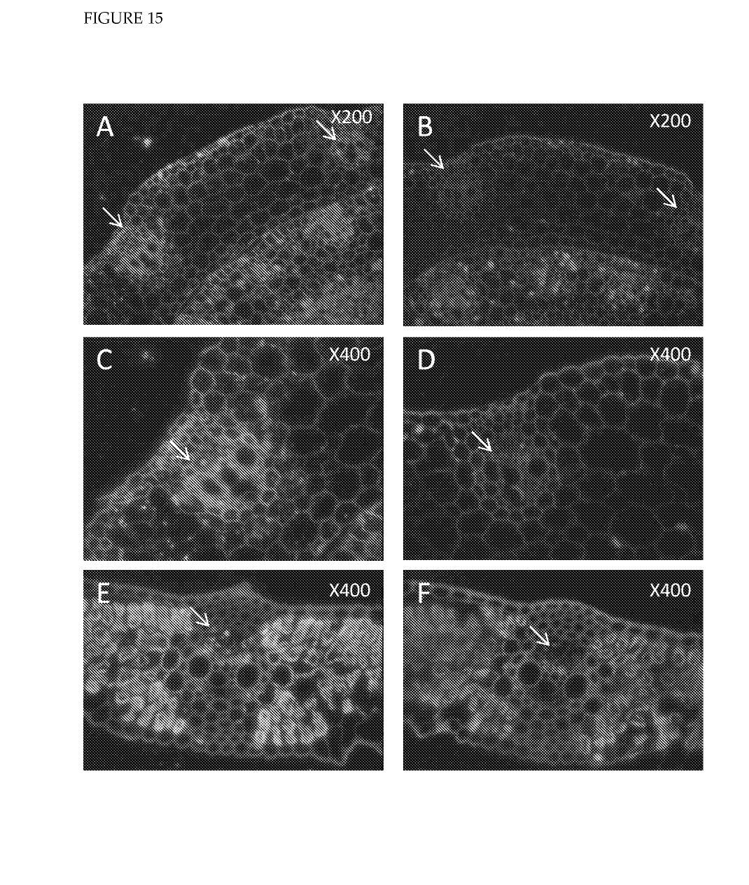

The results of the immunolocalisation experiments are shown in FIG. 15.

Those skilled in the art will appreciate that the invention described herein is susceptible to variations and modifications other than those specifically described. It is to be understood that the invention includes all such variations and modifications. The invention also includes all of the steps, features, compositions and compounds referred to, or indicated in this specification, individually or collectively, and any and all combinations of any two or more of the steps or features.

Also, it must be noted that, as used herein, the singular forms “a”, “an” and “the” include plural aspects unless the context already dictates otherwise.

1. A method for modulating the rate and/or extent of secondary cell wall deposition in a plant cell, the method comprising modulating the expression of a class II homeodomain/leucine zipper (HD-Zip) polypeptide in the plant cell.

2-3. (canceled)

4. The method of claim 1 wherein the class II HD-Zip polypeptide comprises an amino acid sequence selected from the group consisting of: the amino acid sequence set forth in SEQ ID NO: 1 and an amino acid sequence which is at least 50% identical to SEQ ID NO: 1.

5-7. (canceled)

8. The method of claim 1 wherein modulating the rate and/or extent of secondary cell wall deposition in the plant cell comprises modulating the expression of one or more secondary cell wall biosynthetic enzymes in the plant cell.

9-13. (canceled)

14. The method of claim 1 wherein modulating the rate and/or extent of secondary cell wall deposition in the plant cell comprises modulating the rate and/or extent of lignin deposition in the cell wall of the plant cell.

15. (canceled)

16. The method of claim 1 wherein modulating the rate and/or extent of secondary cell wall deposition further effects a modulation in the size of the plant cell as measured in at least one dimension.

17-20. (canceled)

21. A genetically modified plant cell comprising a modulated rate and/or extent of secondary cell wall deposition relative to an unmodified form of the cell, wherein modulation of the rate and/or extent of secondary cell wall deposition is effected by modulation of the expression of a class II homeodomain/leucine zipper (HD-Zip) polypeptide in the genetically modified cell, relative to an unmodified form of the cell.

22-23. (canceled)

24. The cell of claim 21 wherein the class II HD-Zip polypeptide comprises an amino acid sequence selected from the group consisting of: the amino acid sequence set forth in SEQ ID NO: 1 and an amino acid sequence which is at least 50% identical to SEQ ID NO: 1.

25-27. (canceled)

28. The cell of claim 21 wherein the modulated rate and/or extent of secondary cell wall deposition in the plant cell comprises modulation of the expression of one or more secondary cell wall biosynthetic enzymes in the plant cell.

29-33. (canceled)

34. The cell of claim 21 wherein the modulated rate and/or extent of secondary cell wall deposition in the plant cell comprises modulation of the rate and/or extent of lignin deposition in the cell wall of the plant cell.

35. (canceled)

36. The cell of claim 21 wherein the size of the plant cell, as measured in at least one dimension, is also modulated relative to an unmodified form of the cell.

37-40. (canceled)

41. The cell of claim 21, wherein the cell is part of a plant, an organ of the plant or at least one tissue of the plant.

42. The cell of claim 21, wherein the cell is part of a plant cell culture or a plant tissue culture.

43-55. (canceled)

56. A method for determining and/or predicting the rate and/or extent of secondary cell wall deposition in a plant, or a part, organ, tissue or cell thereof, the method comprising determining the expression of a class II homeodomain/leucine zipper (HD-Zip) polypeptide in the plant or a part, organ, tissue or cell thereof.

57-58. (canceled)

59. The method of claim 56 wherein the class II HD-Zip polypeptide comprises an amino acid selected from the group consisting of: the amino acid sequence set forth in SEQ ID NO: 1 and an amino acid sequence which is at least 50% identical to SEQ ID NO: 1.

60-62. (canceled)

63. The method of claim 56 wherein determining and/or predicting the rate and/or extent of secondary cell wall deposition in the plant or a part, organ, tissue or cell thereof comprises determining and/or predicting the expression of one or more secondary cell wall biosynthetic enzymes in the plant or a part, organ, tissue or cell thereof.

64-68. (canceled)

69. The method of claim 56 wherein determining and/or predicting the rate and/or extent of secondary cell wall deposition in a plant or a part, organ, tissue or cell thereof comprises determining and/or predicting the rate and/or extent of lignin deposition in a cell wall of the plant or a part, organ, tissue or cell thereof.

70. (canceled)

71. The method of claim 56 wherein determining the expression of a class II homeodomain/leucine zipper (HD-Zip) polypeptide in the plant or a part, organ, tissue or cell thereof further determines or predicts the size of one or more cells, as measured in at least one dimension, in the plant or a part, organ, tissue or cell thereof.

72-75. (canceled)
